(19) United States
(12) Patent Application Publication
(10) Pub. No.: US 2003/0003086 A1
(43) Pub. Date: Jan. 2, 2003

(54) Modulation of MHC class I antigen presentation

(76) Inventors: Kenneth L. Rock, Chestnut Hill, MA (US); Alfred L. Goldberg, Chestnut Hill, MA (US)

Correspondence Address: J. PETER FASSE, Fish & Richardson P.C., 225 Franklin Street, Boston, MA, 02110-2804, US

(21) Appl. No.: 10/113,085

(22) Filed: Apr. 1, 2002

Related U.S. Application Data
(60) Provisional application No. 60/280,669, filed on Mar. 30, 2001.

(51) Int. Cl. A61K 48/00; C12N 5/08; C12N 15/86

(52) U.S. Cl. 424/93.21; 435/455; 435/366; 435/456

(57) ABSTRACT

The invention is based on the discovery that the cytosolic metalloendopeptidase, thimet oligopeptidase (TOP, EC3.4.25.15) has been found to play a key role in modulating levels of MHC class I-presented peptides, and in doing so, modulates the extent of antigen presentation on cell surfaces. Thus, compositions that affect the presence and/or levels of TOP, and processes to control the presence and/or levels of TOP, can be utilized to modulate antigen presentation on cell surfaces, and thereby enhance the acquisition of cell-based immunity, or alternatively, enhance cell-mediated killing of undesirable cells.

### CROSS-REFERENCE TO RELATED APPLICATIONS

This application claims priority from U.S. Provisional Patent Application Serial No. 60/280,669, filed on Mar. 30, 2001, which is incorporated herein by reference in its entirety.

### STATEMENT AS TO FEDERALLY SPONSORED RESEARCH

The U.S. Government has certain rights in this invention pursuant to Grant No. AI2024 awarded by the National Institutes of Health.

### TECHNICAL FIELD

This invention relates to modulation of Major Histocompatibility Complex (MHC) class I antigen presentation.

### BACKGROUND

In higher vertebrates, an important function of intracellular protein degradation is to generate the small fragments of cell and foreign proteins that are presented to the immune system on surface MHC class I molecules (Rock et al. Annual Rev. Immunol., 1999, 17:739-79). Most of these 8-11 residue peptides are generated by 26S proteasomes during intracellular protein breakdown. Accordingly, inhibitors of the proteasome block MHC class I antigen presentation and suppress T cell responses against various antigens (Craiu et al., J Biol. Chem., 1997, 272(20):13437-45; Cerundolo et al. European Journal of Immunology, 1997, 27:336-41; Rock, K.L. et al. Cell, 1994, 78(5):761-71). Once generated, the antigenic peptides are transported by the transporter associated with antigen processing (TAP) complex from the cytosol into the endoplasmic reticulum (ER), where they bind to MHC class I molecules and are then transported to the cell surface (Pamer et al., Ann. Rev. Immunol., 1998, 16:323-58).

In degrading polypeptides, proteasomes generate peptides ranging from 3-25 residues in length (Kisselev et al., J Biol. Chem., 1998. 273(4):1982-1989; Kisselev et al., J Biol. Chem., 1999, 274(6):3363-71). Such peptides however, cannot be found in the cytosol (Falk et., Nature, 1991, 351(6324):290-296). Efficient breakdown of proteasome products is essential to allow the recycling of amino acids in synthesis of new proteins, but this process is also important in preventing the build up of proteolytic fragments that might interfere with critical protein-protein interactions in the cell. The enzymes responsible for this rapid degradation have not been identified, nor is it clear how the MHC-presented peptides can escape this fate and serve in antigen presentation.

It had long been assumed that the proteasome releases the mature epitopes directly (Niedermann et al., J Exp. Med., 1997, 186:209-220; Lucchiari-Hartz et al., J Exp. Med., 2000, 191:239-252.). However, there is growing evidence that antigenic peptides may be released by 26S proteasomes as longer precursors that undergo trimming by aminopeptidases in the cytosol or ER to yield the presented epitopes (Paz et al., Immunity, 1999, 11:241-251; Craiu et al., Proc. Nat. Acad. Sci. USA, 1997, 94:10850-10855; Beninga et al. J Biol. Chem., 1998, 273:18734-18742; Mo et al., J Immunol, 1999, 163:5851-5859; Stoltze et al., Nature Immunol., 2000, 1:413-418). It is noteworthy that the immune modifier interferon-γ, which enhances class I-presentation, has been shown to induce the leucine amino peptidase, the cytosolic peptidase most active in trimming longer precursors to MHC-presented epitopes. Thus, the role of peptide metabolism in the cytosol in determining the extent of antigen presentation is of interest.

### SUMMARY

The invention is based on the discovery that the cytosolic metalloendopeptidase, thimet oligopeptidase (TOP, EC3.4.25.15), plays a key role in modulating levels of MHC class I-presented peptides, and in doing so, modulates the extent of antigen presentation on cell surfaces. Thus, compositions that affect the activity, presence, and/or levels (expression) of TOP, and processes to control the presence an&or levels of TOP, can be utilized to modulate antigen presentation on cell surfaces. When TOP levels or activity are decreased, there is an enhancement of the acquisition of cell-based immunity, or alternatively, enhancing cell-mediated killing of undesirable cells. An increase in TOP levels can facilitate blocking of immune responses. The invention therefore relates to compositions that modulate, and processes for modulating, MHC class I antigen presentation, as well as screening methods for identifying such modulators. As such, these compositions and processes relate to regulation of the immune system and relate to therapeutic applications involving regulation of the immune system.

In some embodiments, vectors are used to transfect cells that are to be introduced or implanted into a host, such as an animal or human patient in need of the cells. The cells (recombinant cells), can be for example, stem cells or bone marrow stromal cells, e.g., stem cells that have been engineered to include a desired gene that is to be expressed in the host. A cell containing such an introduced gene encoding TOP, PSA, TOP fragment, or PSA fragment is referred to as containing an exogenously derived nucleic acid. TOP polypeptides include wild-type TOP proteins (full-length), and variants of TOP that have at least one TOP biological activity. Such activities include the ability to bind to a naturally occurring TOP substrate and TOP enzymatic activity. Progeny of such cells are also identified as containing an exogenously derived nucleic acid. If the nucleic acid is not identical to a TOP or PSA gene that is endogenous to the cell, the nucleic acid is termed “heterologous.” To reduce the immunogenic effect of these cells, they can also be engineered to include the TOP gene, to overexpress TOP and thereby ensure that fewer, or no, antigenic peptides appear on the surface of the engineered cells once implanted into the patient. Overexpression refers to an amount of expression (mRNA or polypeptide) that is increased relative to expression in a wild-type (e.g., non-recombinant) cell. The wild-type cell is generally a cell from the same lineage or cell type as the recombinant cell. In some embodiments, increased activity of TOP is measured. In this case, the activity of TOP is increased relative to a wild-type cell. In other embodiments of the invention, underexpression or decreased activity of TOP is desirable.

In general, the invention features a cell including an overexpressed amount of a cytosolic metallopeptidase enzyme, e.g., thimet oligopeptidase (TOP) and/or purmycin-sensitive aminopeptidase (PSA) compared with a wild type (e.g., unengineered) cell. The invention also includes a cell having an effective amount of a cytosolic metallopeptidase enzyme, e.g., TOP, to reduce the level of one or more MHC class I-presented antigenic peptides otherwise present in the cell.

The invention includes a recombinant cell containing an exogenously derived nucleic acid coding for a thimet oligopeptidase (TOP) polypeptide, such that the TOP polypeptide is overexpressed in the cell compared to a wild-type cell from which the recombinant cell was derived. The nucleic acid coding for the TOP polypeptide can be a heterologous nucleic acid sequence. The TOP polypeptide can be a full-length TOP polypeptide or a fragment of a full-length TOP polypeptide, e.g., that has TOP activity. In some embodiments, the sequence is introduced into the cell by transfection with a vector, e.g., a viral vector. The cell can be, e.g., a stem cell or a stromal cell. In other embodiments, the cell expresses an amount of TOP effective to reduce the level of one or more MHC class I-presented antigenic peptides present in the cell. In yet another embodiment, the cell expresses an amount of TOP effective to reduce the T-cell response otherwise produced in a mammal to the cell. In some aspects of the invention, the cell is transfected with a vector (e.g., a viral vector) comprising a nucleic acid encoding PSA, such that PSA is overexpressed in the cell.

In some embodiments, the invention includes a method for reducing the antigenicity of a cell. The method includes the step of introducing into the cell a nucleic acid that expresses a TOP polypeptide. The cell can be, e.g., a stem cell or a stromal cell. In another embodiment. The invention also includes a method for modulating an antigenic response (increasing or decreasing the antigenic response) in a mammal. The method includes the step of administering to the mammal cells (e.g., stem cells or stromal cells) transfected with a nucleic acid encoding a TOP polypeptide. In some embodiments, the cell is removed from the mammal, transfected in vitro, and reintroduced into the mammal. The invention includes a method of screening a test compound for its ability to serve as an immunomodulatory agent, which includes the steps of obtaining a test compound; contacting the test compound with a thimet oligopeptidase (TOP); and assessing the ability of the test compound to interact with TOP, such that a test compound that interacts with TOP is a candidate immunomodultory agent. The interaction can be, e.g., direct binding of the test compound to TOP or one such that TOP expression or activity is altered (TOP expression or activity is increased or decreased). In some embodiments, the cell overexpresses TOP and puromycin-sensitive aminopeptidase (PSA).

In some aspects, the invention includes a method of modulating an antigenic response to a cell in a mammal. The method includes the step of inducing overexpression of TOP in the cell and introducing the cell into the mammal. In some embodiments of this method, PSA is also overexpressed in the cell. The cell can be transfected with a nucleic acid encoding a TOP protein or polypeptide and the cell can be, e.g., a stem cell or a stromal cell.

The invention includes a method for modulating an antigenic response in a mammal. The method includes the step of administering to cells or tissue of a mammal in vivo a composition containing a nucleic acid encoding a TOP polypeptide. In some aspects, the invention is a method for modulating an antigenic response in a mammal. The includes the step of administering to the mammal an amount of a TOP inhibitor to the mammal that is effective to modulate the antigenic response. The additional step can be included of administering to the mammal cells that contain enhanced levels (relative to a wild-type cell or control) of antigenic peptides. In some embodiments of the invention, the method involves administering at least two inhibitors of TOP to the mammal. In another embodiment, at least one TOP inhibitor and at least on PSA inhibitor are administered.

In another aspect, the invention includes a method for identifying an antigen resistant to thimet oligopeptidase (TOP) degradation. The method includes the steps of selecting a first antigen that is degraded by TOP, synthesizing a second antigen homologous to the first antigen except for one or more amino acid substitutions, additions, or deletions relative to the first antigen, and assessing the degradation of the second antigen to thimet oligopeptidase degradation compared to degradation of the first antigen, such that a decrease in the amount of degradation of the second antigen compared to the amount of degradation of the first antigen indicates that the second antigen is resistant to TOP degradation. The second antigen can, e.g., bind MHC Class I molecules or T-cell receptors.

The invention includes a tissue engineering construct that contains a cell of the invention.

In another embodiment the invention includes a method of screening test compounds for their ability to modulate thimet oligopeptidase (TOP) expression or activity. The method includes the steps of obtaining a test compound, contacting the test compound with TOP or a nucleic acid that encodes TOP, and assessing the ability of the test compound to interact with TOP or the nucleic acid that encodes TOP, such that a test compound that interacts with TOP or a TOP-encoding nucleic acid is a candidate immunomodulatory agent. The invention further includes a method of increasing CD8 T cell immunity. The method includes the step of including in a vaccination a TOP inhibitor in an amount effective to decrease TOP expression or activity. The vaccination method can include the use of, e.g., treated tumor cells, antigen bearing/pulsed dendritic cells, or injection of a viral vector. The invention also includes compositions used in such vaccination protocols.

As used herein, the terms “TOP” and “THOP” refer to thimet oligopeptidase.

The invention provides several advantages. Compositions and processes that modulate levels of MHC class I-presented peptides are useful for either reducing or enhancing cytolytic immune responses. The suppression of such immune responses is an important requirement for successful transplantation of organs and for many emerging cellular therapies, such as those involving introduction of foreign stem cells into a mammal for therapeutic purposes. Another advantage of this approach is that it does not involve a general suppression of the recipient's immune system. Furthermore, it cannction synergistically with known immunosuppressive drugs. Enhancing immune responses enhances the success of vaccination and stimulation of the immune system's capacity to counter cancer and various viruses. Thus, inhibition of TOP and/or PSA expression or activity is useful for enhancing an immune response. A further advantage of certain aspects of compositions, and processes, that modulate levels of MHC class I-presented peptides, is that where introduction and/or exposure to chemical inhibitors of TOP impractical (i.e., where they can not enter cells), certain cellular-based approaches to modulating MHC class I-presented peptides provide alternative means for modulation of antigen presentation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A, 1B, 1C, and 1D are graphs showing the effect of TOP transfection on inhibition of MHC class I presentation of cytosolic peptides.

FIGS. 2A, 2B, and 2C are graphs showing the effect of TOP transfection on inhibition of MHC class I presentation of a full-length protein.

FIGS. 3A, 3B, and 3C are graphs showing inhibition of TOP and MHC class I presentation in normal cells, and of overexpression of TOP and the presentation of peptides generated in the endoplasmic reticulum or endocytic compartments.

FIGS. 4A, 4B, 4C, and 4D are graphs showing TOP inhibition and the appearance of MHC class I molecules on the cell surface.

FIGS. 5A and 5B together are a representation of the nucleic acid sequence of the human TOP gene (SEQ ID NO: 1) from Genbank Accession No. BC000135.

FIG. 6 is a representation of the amino acid sequence of human TOP (SEQ ID NO:2) from Genbank Accession No. BC000135.

### DETAILED DESCRIPTION

Compositions that affect the presence, activity, and/or levels of TOP, and processes to control the presence, activity, and/or levels of TOP, can be utilized to modulate antigen presentation on cell surfaces, and thereby enhance the acquisition of cell-based immunity, or alternatively, enhance cell-mediated killing of undesirable cells (e.g., tumor, viral). The invention relates to compositions that modulate, and processes for modulating, MHC class I antigen presentation, as well as processes for identifying modulators. As such, these compositions and processes are useful for regulating the immune system and for therapeutic applications involving regulation of the immune system.

The composition of the invention for modulating antigen presentation include polypeptides (e.g., TOP, PSA, or a fragment of a full-length TOP or PSA), nucleic acids (e.g., encoding TOP, PSA, or an active fragment of a TOP or PSA), vectors, and host cells. The methods of modulating antigen presentation utilize these compositions, as well as other reagents and assay methodologies to assess TOP presence and/or antigen presence. The following description provides further detail on the manner of making and using the invention and its various embodiments.

Methods of Use

The new methods can be used to reduce or enhance cytolytic immune responses. The suppression of such immune responses is an important requirement for successful transplantation of organs and for many cellular therapies, such as those involving introduction of foreign stem cells or bone marrow stromal cells from donors other than the host or patient. In addition, the new techniques can also be used in methods to enhance vaccination protocols and to stimulate the immune system's capacity to counter cancer and various viruses.

Cytolytic immune responses are based upon the ability of CD8-positive lymphocytes to recognize abnormal cells (e.g. foreign or viral-infected cells) through the nature of antigenic peptides displayed on MHC-Class I molecules. Various immune suppression strategies are based upon approaches to interfere with the functioning of these cell's recognition and activation mechanisms to prevent the interactions between the T-cells and the antigen-presenting cells, etc. The new methods utilize the effect of TOP to control the level of antigenic peptides presented on the surface of cells. Thus, controlling the expression and/or activity of TOP can be used to control the number of antigenic peptides expressed on the surface of cells, and thus the immunogenic effect of the cells when introduced into a host or patient. In this manner, CD8 T cell immunity can be modulated.

The new methods are based upon the discovery that TOP functions in the pathway for antigen presentation in all cells, and the demonstration (as evidenced by the Examples below) that by enhancing the TOP expression or activity in cells, one can inhibit or prevent the normal appearance of antigenic peptides on surface MHC-Class I molecules. The proteasome is the main source of these antigenic peptides (see, e.g., Rock and Goldberg, 1999, Ann Rev. immunol. 17:739-779). During the course of protein breakdown, this structure generates small peptides (3-24 amino acids long), which are generally rapidly broken down by cellular peptidases to amino acids, which the cell can reuse in protein synthesis. A small fraction of these peptides are somehow able to escape such destruction, and are taken up into the endoplasmic reticulum, where they bind to MHC-Class I molecules and are delivered to the cell surface. The present invention is based on the discovery that antigenic peptides are also susceptible to rapid digestion and that the enzyme responsible for this processing is TOP. Previously, the function of this enzyme was unknown. These findings and experiments on intact cells indicate that when antigenic peptides or longer variants are released from proteasomes, a large fraction (perhaps most) are degraded in the cytosol, and thus, few are able to bind to the TAP transporter in the endoplasmic reticulum and serve in antigen presentation.

It has also been discovered that if TOP is overexpressed in cells by transfection of the gene for thymet oligopeptidase using viral vectors, degradation of antigenic peptides in the cytosol is enhanced. Consequently, the antigenic peptides arising by proteasome activity were particularly short-lived and unable to reach the MHC-molecules in the ER. These model experiments demonstrated a general reduction in the surface content of MIC-Class I molecules, and this effect was shown to be highly selective (e.g., MHC Class II responses, which elicit antibody responses, were not affected). Thus, by overproduction of this single protein, for example in foreign stem cells, it is possible to greatly reduce the ability of the host's immune system to recognize and destroy the foreign cells. One major advantage of this approach is that it does not involve a general suppression of the recipient's immune system, and it will function synergistically with known immunosuppressive drugs.

These discoveries also led to the invention of a new strategy for enhancing antigen presentation by cells. This approach is important in vaccination regimens or in efforts to boost the host's immune system's ability to destroy cancers or other viral diseases. Chemical inhibitors of TOP are known, but they generally cannot enter the cell cytosol. Using hypertonic lysis of pinosomes to load TOP inhibitors into the cytoplasm of cells with these inhibitors, it is shown herein that inhibition of this enzyme in normal cells leads to less intracellular destruction of antigenic peptides or their precursors, and resulted in greater amounts of antigenic peptides on surface MHC molecules (Example 5, infra). Similar methods can be used to inhibit PSA expression or acitivity.

A variety of standard approaches exist for the identification of such inhibitors by screening of chemical libraries or natural product libraries, and by rational drug design based upon the known specificity of this enzyme and its known mechanism of actions. Any such inhibitors of TOP can be used in the new methods to enhance the acquisition of cell-based immunity or to enhance cell-mediated killing of tumors or virally-infected cells. This approach is useful together with a variety of immunization methods.

It has also been discovered that the breakdown of FAPGNYPAL (SEQ ID NO: 10; Sendai Virus NP) was particularly rapid and not affected by inhibitors of TOP. Because the degradation of this peptide also involved metallopeptidases, and was inhibited up to 70% by puromycin or bestatin, cytosolic aminopeptidase, puromycin-sensitive aminopeptidase (PSA) is implicated in the degradation of this peptide. In addition, FAPGNYPAL was particularly susceptible to degradation by pure PSA at concentrations similar to those found in HeLa cells. It has also been found that degradation of RGYVYQGL (SEQ ID NO: 12; vesicular stomatitis virus nucleoprotein cytotoxic T cell epitope 52-59; Stoltze et al., 2000, Nature Immunol. 1:413-418) in HeLa extracts is not affected by inhibitors of TOP. Therefore, PSA is involved in the hydrolysis and degradation of a subset of antigenic peptides, not degraded rapidly by TOP. Thus, the techniques described herein for modulating the expression and/or activity of TOP can be used to modulate the expression and/or activity of PSA.

The methods of therapeutic application of the invention utilize embodiments mentioned herein, as well as other reagents and methodologies to provide the therapy of interest. The compositions and methods for modulating immune response described herein can be used for immunosuppressive therapy, for example, suppressing autoimmune response in inflammatory disease, tissue destruction, exogenous tissue engineering, or organ transplant rejection. They can also be used in immunostimlulative therapy, for example, vaccines, cellular targeting (e.g., enhancing immune response for tumor or infected viral cells).

The following description provides further detail on the manner of making and using the invention and its various embodiments for methods of therapeutic application, including immunomodulation.

For use of the new techniques in conjunction with vaccines as described herein, immunization can be assessed in animal models, such as rat or mouse, by administration of cells in which TOP expression and/or activity is suppressed or inhibited, to the subject. Inhibitors of TOP include peptides, antibodies, and small molecules, for example, Cpp-AAFpAb, and monoclonal antibodies such as IVD6, MG132 (Peptides International, Louisville, Ky.), PMSF (Sigma, St. Louis, Mo.), and E64 (Sigma, St. Louis, Mo.). Antibodies can inhibit TOP or can be useful to deplete TOP from cell extracts. Assessment of response can be ascertained in a variety of ways, including, for example, analysis of humoral immune response or analysis of cellular immune response. The former technique utilizes analysis of blood plasma samples for different types of nucleosome reactive antibodies, which may be quantified using a variety of methods, including, for example, ELISA-based systems, staining and FACScan (Becton Dickinson, Mountainview, Calif.). The latter technique examines cellular toxicity in in vitro assays.

Administration of such cells and the corresponding vaccines can be by intraperitoneal routes. In addition, vaccines can be administered intravenously, intramuscularly, transmucosally, or subcutaneously. These modes of administration can also be combined. For example, the first administration can be transmucosal and the subsequent administration can be intraperitoneal.

The invention can also be used in combination with other vaccine methods known in the art such as those that use treated-tumor cells, antigen bearing/pulsed dendritic cells (U.S. Ser. No. 20020019047; Hsu, F. J. et al., National Med., 2:52-58, 1996; Celluzzi et al., J. Exp. Med., 183:283-287, 1996; Van Tsai et al., Timmerman et al., 2002, Crit. Rev. Immuunol. 18:65-75, 1998; Blood 99(5):1517-2615), or injection of viral vector protocols.

The antigen presented in a composition that is combined with inhibition of TOP and/or PSA expression or activity comprises a composition that can include whole cells, hapten- or virus- or cytokine-modified cells, a viral lysate of cells, cell lysate, cell extract, RNA (e.g., RNA-pulsed dendritic cells), tumor-derived exosomes, a purified antigen, a recombinantly produced antigen, a synthetic antigen (e.g., synthesized chemically), a combination of antigens (polyvalent), DNA (e.g., which when administered, produces one or more antigens in cells which take up and express the DNA), DNA encoding an anti-idiotype antibody which mimics an epitope of an antigen (e.g., which when administered, produces a peptide or polypeptide which mimics antigen in cells that take up and express the DNA), one or more antigens presented by antigen presenting cells, and a combination thereof. An antigen may be from autologous or allogeneic or semi-allogeneic (expresses both allogeneic and syngeneic determinants) tumor. Such cells and antigens are typically tumor cells or antigens that are associated with tumors.

Polypeptides, Nucleic Acids, and Inhibitors

An “isolated” nucleic acid molecule is one that is separated from other nucleic acid molecules that are present in the natural source of the nucleic acid. In general, an “isolated” nucleic acid is free of sequences (e.g., protein encoding sequences), which naturally flank the nucleic acid (i.e., sequences located at the 5′ and 3′ ends of the nucleic acid) in the genomic DNA of the organism from which the nucleic acid is derived. For example, in various embodiments, the isolated TOP nucleic acid molecule can contain less than about 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

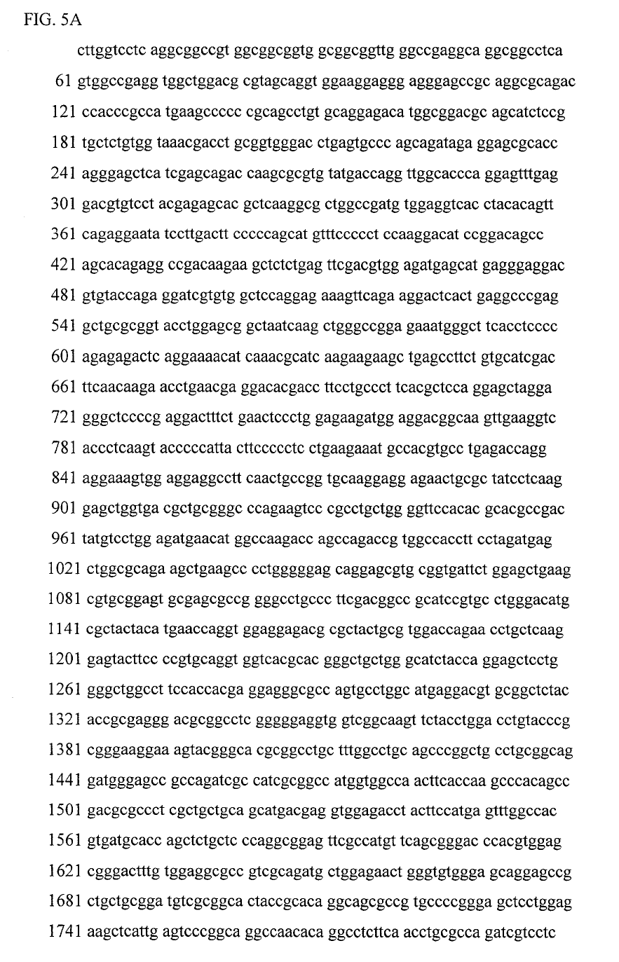

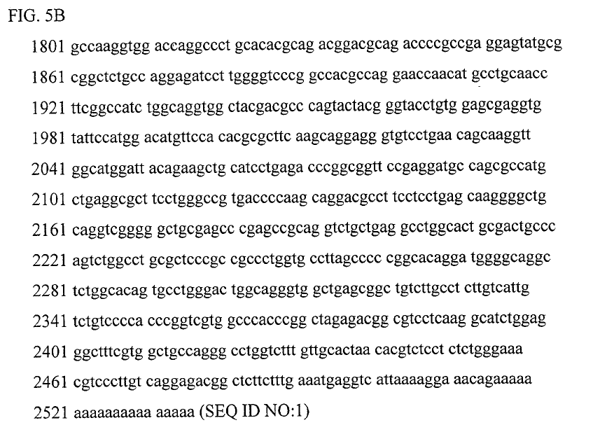

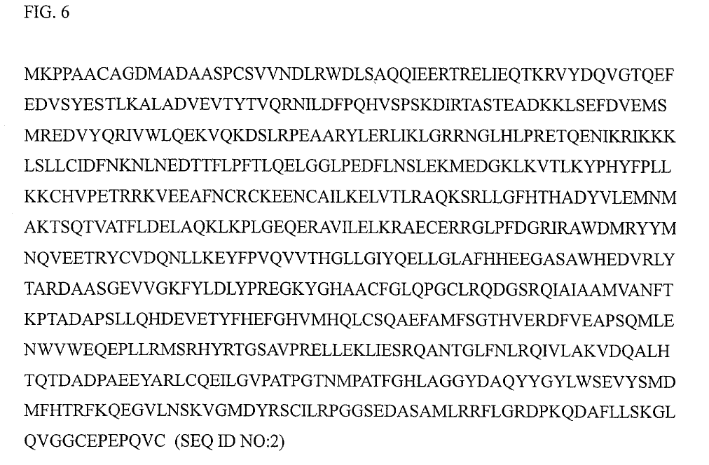

A nucleic acid molecule of the present invention, e.g., a nucleic acid molecule having the nucleotide sequence of SEQ ID NO: 1 (FIGS. 5A and 5B), or a complement of any of these nucleotide sequences, can be isolated using standard molecular biology techniques and the sequence information provided herein. Using all or portion of the nucleic acid sequences of SEQ ID NO: 1, as a hybridization probe, TOP nucleic acid molecules can be isolated using standard hybridization and cloning techniques (e.g., as described in Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989).

A nucleic acid of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to TOP nucleotide sequences can be prepared by standard synthetic techniques, e.g., using an automated DNA synthesizer.

The isolated nucleic acid molecules of the invention can also include a nucleic acid molecule that is a complement of the nucleotide sequence shown in SEQ ID NO:1, or a portion thereof. A nucleic acid molecule that is complementary to a given nucleotide sequence is one which is sufficiently complementary to the given nucleotide sequence that it can hybridize to the given nucleotide sequence thereby forming a stable duplex.

Moreover, the nucleic acid molecules of the invention can comprise only a portion of a nucleic acid sequence encoding TOP, for example, a fragment that can be used as a probe or primer, or a fragment encoding a biologically active portion of TOP. The nucleotide sequence determined from the cloning of the murine and human TOP genes allows for the generation of probes and primers designed for use in identifying and/or cloning TOP homologs in other cell types, e.g., from other tissues, as well as TOP homologs from other mammals. The probe/primer typically comprises a substantially purified oligonucleotide. The oligonucleotides typically comprise a region of nucleotide sequence that hybridizes under stringent conditions to at least about 12, e.g., about 25, or about 50, 75, 100, 125, 150, 175, 200, 250, 300, 350, or 400 consecutive nucleotides of the sense or antisense sequence of SEQ ID NO: 1, or of a naturally occurring mutant of SEQ ID NO: 1.

Probes based on the TOP nucleotide sequence can be used to detect transcripts or genomic sequences encoding the same or related proteins. The probe comprises a label group attached thereto, e.g., a radioisotope, a fluorescent compound, an enzyme, or an enzyme co-factor. Such probes can be used as a part of a diagnostic test kit for identifying cells or tissue which mis-express a TOP protein, such as by measuring a level of a TOP-encoding nucleic acid in a sample of cells from a subject, e.g., detecting TOP mRNA levels or determining whether a genomic TOP gene has been mutated or deleted.

A nucleic acid fragment encoding a “biologically active portion of TOP” can be prepared by isolating a portion of SEQ ID NO: 1, which encodes a polypeptide having a TOP biological activity, expressing the encoded portion of TOP (e.g., by recombinant expression in vitro) and assessing the activity of the encoded portion of TOP.

The invention further encompasses the use of nucleic acid molecules that differ from the nucleotide sequence of SEQ ID NO: 1, due to degeneracy of the genetic code, but that still encode the same TOP protein as that encoded by the nucleotide sequence shown in SEQ ID NO: 1, e.g., that encode the amino acid sequence of SEQ ID NO:2.

In addition to the TOP nucleotide sequence shown in SEQ ID NO: 1, it will be appreciated by those skilled in the art that DNA sequence polymorphisms that lead to changes in the amino acid sequences of TOP may exist within a population. Such genetic polymorphisms in the TOP gene may exist among individuals within a population due to natural allelic variation. As used herein, the terms “gene” and “recombinant gene” refer to nucleic acid molecules comprising an open reading frame encoding a TOP protein, preferably a mammalian TOP protein. Such natural allelic variations can typically result in 1-5% variance in the nucleotide sequence of the TOP gene. Any and all such nucleotide variations and resulting amino acid polymorphisms in TOP that are the result of natural allelic variation and that do not alter the functional activity of TOP are intended to be within the scope of the invention. The recombinant gene can code for a TOP protein, PSA protein, or an active fragment of a TOP or PSA. Inactive TOP proteins, PSA proteins, TOP polypeptide, or PSA polypeptides are useful in some embodiments of the invention, e.g., to inhibit TOP activity.

Moreover, the use of nucleic acid molecules encoding TOP proteins from other species (TOP homologs), that have a nucleic acid sequence that differs from that of the murine gene or human gene, are within the scope of the invention. Nucleic acid molecules corresponding to natural allelic variants and homologs of the TOP cDNA of the invention can be isolated based on their identity to the TOP nucleic acids disclosed herein using the murine cDNAs, or a portion thereof, as a hybridization probe according to standard hybridization techniques under stringent hybridization conditions. For example, a soluble TOP cDNA can be isolated based on its identity to murine or human TOP.

Accordingly, in another embodiment, an isolated nucleic acid molecule of the invention is at least 300 (325, 350, 375, 400, 425, 450, 500, 550, 600, 650, 700, 800, 900, 1000, or 1200) nucleotides in length and hybridizes under stringent conditions to the nucleic acid molecule comprising the nucleotide sequence, e.g., the coding sequence, of SEQ ID NO: 1 or a complementary sequence thereof. As used herein, the term “hybridizes under stringent conditions” describes conditions for hybridization and washing under which nucleotide sequences at least 60% (65%, 70%, in general 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A non-limiting example of stringent hybridization conditions is hybridization in 6X sodium chloride/sodium citrate (SSC) at about 45° C., followed by one or more washes in 0.2X SSC, 0.1% SDS at 50-65° C. In general, an isolated nucleic acid molecule of the invention that hybridizes under stringent conditions to the sequence of SEQ ID NO: 1 or a complementary sequence, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the TOP sequence that may exist in the population, the skilled artisan will further appreciate that changes can be introduced by mutation into the nucleotide sequences disclosed herein, thereby leading to changes in the amino acid sequence of the encoded TOP protein, without altering the functional ability of the TOP protein. For example, one can make nucleotide substitutions leading to amino acid substitutions at “non-essential” amino acid residues. A “non-essential” amino acid residue is a residue that can be altered from the wild-type sequence of TOP without altering the biological activity of the encoded polypeptide, whereas an “essential” amino acid residue is required for biological activity. For example, amino acid residues that are conserved among the TOP proteins of various species are predicted to be particularly unamenable to alteration.

Accordingly, another aspect of the invention pertains to nucleic acid molecules encoding TOP proteins that contain changes in amino acid residues that are not essential for activity. Such TOP proteins differ in amino acid sequence from those disclosed herein yet retain biological activity. In one embodiment, the isolated nucleic acid molecule includes a nucleotide sequence encoding a protein that includes an amino acid sequence that is at least about 65%, 75%, 85%, 95%, or 98% or more identical to the amino acid sequence of SEQ ID NO:2.

An isolated nucleic acid molecule encoding a TOP protein having a sequence which differs from that disclosed herein can be created by introducing one or more nucleotide substitutions, additions or deletions into the nucleotide sequence disclosed herein such that one or more amino acid substitutions, additions or deletions are introduced into the encoded protein. Mutations can be introduced by standard techniques, such as site-directed mutagenesis and PCR-mediated mutagenesis. In general, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in TOP is generally replaced with another amino acid residue from the same side chain family. Alternatively, mutations can be introduced randomly along all or part of a TOP coding sequence, such as by saturation mutagenesis, and the resultant mutants can be screened for TOP biological activity to identify mutants that retain activity. Following mutagenesis, the encoded protein can be expressed recombinantly and the activity of the protein can be determined.

The present invention also encompasses antisense nucleic acid molecules, i.e., molecules that are complementary to a sense nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire TOP coding strand, or to only a portion thereof, e.g., all or part of the protein coding region (or open reading frame). An antisense nucleic acid molecule can be antisense to a noncoding region of the coding strand of a nucleotide sequence encoding TOP. The noncoding regions (“5′ and 3′ untranslated regions”) are the 5′ and 3′ sequences, which flank the coding region and are not translated into amino acids.

Given the coding strand sequences encoding TOP disclosed herein (e.g., SEQ ID NO: 1), antisense nucleic acids of the invention can be designed according to the rules of Watson and Crick base pairing. The antisense nucleic acid molecule can be complementary to the entire coding region of TOP mRNA, but is generally an oligonucleotide which is antisense to only a portion of the coding or noncoding region of TOP mRNA. For example, the antisense oligonucleotide can be complementary to the region surrounding the translation start site of TOP mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine,7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding the protein of interest to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection into a cell, such as a stem cell, or into a tissue site or engineered tissue construct. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids. Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, 1988, Nature 334:585-591) can be used to catalytically cleave TOP mRNA transcripts to thereby inhibit translation of TOP mRNA. A ribozyme having specificity for a TOP-encoding nucleic acid can be designed based upon the nucleotide sequence of a TOP cDNA disclosed herein. For example, a derivative of a Tetrahymena L-19 IVS RNA can be constructed in which the nucleotide sequence of the active site is complementary to the nucleotide sequence to be cleaved in a TOP-encoding mRNA. See, e.g., Cech et al. U.S. Pat. No. 4,987,071; and Cech et al. U.S. Pat. No. 5,116,742. Alternatively, TOP mRNA can be used to select a catalytic RNA having a specific ribonuclease activity from a pool of RNA molecules. See, e.g., Bartel and Szostak, 1993, Science 261:1411-1418.

Interfering RNA methods can also be used to modulate the expression of TOP and/or PSA. In these methods, interfering RNA is used as oligonucleotides or as longer sequences to effectively knock out TOP expression. The use of RNA can result in a reduction or elimination of expression of a specific sequence (e.g., TOP or PSA). The method of doublestranded RNA interference (dsRNAi) is based on the interfering properties of doublestranded RNA derived from the coding regions of a gene (Fire et al., Nature, 1998, 391:806-811). RNA can also be used to generate loss-of-function phenotypes (e.g., Kennerdell and Carthew, Cell, 1998, 95:1017-1026; Misquitta and Patterson, Proc. Nat. Acad. Sci. USA, 1999, 96:1451-1456). In one example of this method, complementary sense and antisense RNAs derived from a substantial portion of a gene of interest (e.g., TOP or PSA) are synthesized in vitro. The resulting sense and antisense RNAs are annealed in an injection buffer, and the double-stranded RNA injected or otherwise introduced into an animal or a cell. Progeny of the cell or animal are then inspected for phenotypes of interest (see, e.g., PCT Publication No. WO99/32619), e.g., decreased expression of TOP or PSA. In another embodiment of the method, dsRNA derived from TOP or PSA can be generated in vivo by simultaneous expression of both sense and antisense RNA from appropriately positioned promoters operably fused to subject sequences in both sense and antisense orientations. In yet another embodiment of the method the dsRNA can be delivered to the animal by engineering expression of dsRNA within cells of a second organism that serves as food for the animal.

RNA can be used to inhibit expression of targeted proteins such as TOP or PSA (Caplen et al., 2000, Gene 252:95-105). Thus, cell lines in culture can be manipulated using RNA both to perturb and study the function of subject pathway components and to validate the efficacy of therapeutic strategies that involve the manipulation of this pathway. The method can also be used to generate cells useful for vaccines that have decreased TOP expression or activity. RNA, can also be used to generate loss-of-function phenotypes; which can, in turn, be used, e.g., to determine gene function. Methods relating to the use of RNA to silence genes in C. elegans, Drosophila, plants, and humans are known in the art (Fire et al., 1998, Nature 391:806-811; Fire, 1999, Trends Genet. 15:358-363; Sharp, 2001. Genes Dev. 15:485-490; Hammond et al., 2001, Nature Rev. Genet. 2:110-1119; Tuschl, 2001, Chem. Biochem. 2:239-245 (2001); Hamilton et al., 1999, Science 286:950-952; Hammond et al., 2000, Nature 404:293-296; Zamore et al., 2000, Cell 101:25-33; Bernstein et al., 2001, Nature 409:363-366; Elbashir et al., 2001, Genes Dev. 15:188-200; WO0129058; WO9932619; Elbashir et al., 2001, Nature 411:494-498).

The invention also encompasses nucleic acid molecules that form triple helical structures. For example, TOP gene expression can be inhibited by targeting nucleotide sequences complementary to the regulatory region of the TOP (e.g., the TOP promoter and/or enhancers) to form triple helical structures that prevent transcription of the TOP gene in target cells. See generally, Helene, 1991, Anticancer Drug Des. 6(6):569-84; Helene, 1992, Ann. N.Y. Acad. Sci. 660:27-36; and Maher, 1992, Bioassays 14(12):807-15.

In certain embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAS” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. USA 93: 14670-675.

PNAs of TOP can be used therapeutic and diagnostic applications. For example, PNAs can be used as antisense or antigene agents for sequence-specific modulation of gene expression by, e.g., inducing transcription or translation arrest or inhibiting replication. PNAs of TOP can also be used, e.g., in the analysis of single base pair mutations in a gene by, e.g., PNA directed PCR clamping; as artificial restriction enzymes when used in combination with other enzymes, e.g., S1 nucleases (Hyrup (1996) supra; or as probes or primers for DNA sequence and hybridization (Hyrup (1996) supra; Perry-O'Keefe et al. (1996) Proc. Natl. Acad. Sci. USA 93: 14670-675).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

Isolated TOP Proteins and Anti-TOP Antibodies

One aspect of the invention pertains to isolated TOP proteins, and biologically active portions thereof, as well as polypeptide fragments suitable for use as immunogens to raise anti-TOP antibodies. In one embodiment, native TOP proteins can be isolated from cells or tissue sources by an appropriate purification scheme using standard protein purification techniques. In another embodiment, TOP proteins are produced by recombinant DNA techniques. Alternative to recombinant expression, a TOP protein or polypeptide can be synthesized chemically using standard peptide synthesis techniques.

An “isolated” or “purified” protein or biologically active portion thereof is substantially free of cellular material or other contaminating proteins from the cell or tissue source from which the protein of interest is derived (e.g., TOP), or substantially free from chemical precursors or other chemicals when chemically synthesized. The language “substantially free of cellular material” includes preparations in which the protein is separated from cellular components of the cells from which it is isolated or recombinantly produced. Thus, TOP protein that is substantially free of cellular material includes preparations of TOP protein having less than about 30%, 20%, 10%, or 5% (by dry weight) of non-TOP protein (also referred to herein as “contaminating protein”). When the TOP protein or biologically active portion thereof is recombinantly produced, it is also preferably substantially free of culture medium, i.e., culture medium represents less than about 20%, 10%, or 5% of the volume of the protein preparation. When TOP protein or peptides are produced by chemical synthesis, they are preferably substantially free of chemical precursors or other chemicals, i.e., they are separated from chemical precursors or other chemicals that are involved in the synthesis of the protein. Accordingly, such preparations of TOP protein have less than about 30%, 20%, 10%, 5% (by dry weight) of chemical precursors or non-TOP materials, e.g., chemicals.

Biologically active portions of a TOP protein include peptides comprising amino acid sequences sufficiently identical to or derived from the amino acid sequence of the TOP protein, which include fewer amino acids than the full-length TOP proteins, and exhibit at least one activity of a TOP protein. Typically, biologically active portions comprise a domain or motif with at least one activity of the TOP protein. A biologically active portion of a TOP protein can be a polypeptide, which is, for example, 10, 25, 50, 100 or more amino acids in length. Biologically active polypeptides generally include one or more identified TOP structural domains.

Moreover, other biologically active portions, in which other regions of the protein are deleted, can be prepared by recombinant techniques and evaluated for one or more of the functional activities of a native TOP protein.

TOP proteins generally have or are substantially identical to the amino acid sequences disclosed herein. Certain TOP proteins are substantially identical to those disclosed herein and retain the functional activity of the wild type TOP protein yet differ in amino acid sequence due to natural allelic variation or mutagenesis.

Accordingly, a useful TOP protein is a protein which includes an amino acid sequence at least about 55%, 65%, 75%, 85%, 95%, or 99% identical to the amino acid sequence of SEQ ID NO:2, and retains the functional activity of the TOP protein of SEQ ID NO:2. In one embodiment, the TOP protein retains a functional activity of the TOP protein of SEQ ID NO: 2.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions×100).

The determination of percent homology between two sequences can be accomplished using a mathematical algorithm. A non-limiting example of a mathematical algorithm utilized for the comparison of two sequences is the algorithm of Karlin and Altschul (1990, Proc. Nat'l Acad. Sci. USA 87:2264-2268) modified as in Karlin and Altschul (1993, Proc. Nat'l Acad. Sci. USA 90:5873-5877). Such an algorithm is incorporated into the NBLAST and XBLAST programs of Altschul, et al. (1990, J. Mol. Biol. 215:403-410). BLAST nucleotide searches can be performed with the NBLAST program, score =100, wordlength=12 to obtain nucleotide sequences homologous to TOP nucleic acid molecules of the invention. BLAST protein searches can be performed with the XBLAST program, score=50, wordlength=3 to obtain amino acid sequences homologous to TOP protein molecules of the invention. To obtain gapped alignments for comparison purposes, Gapped BLAST can be utilized as described in Altschul et al., (1997, Nucleic Acids Res. 25:3389-3402). When utilizing BLAST and Gapped BLAST programs, the default parameters of the respective programs (e.g., XBLAST and NBLAST) can be used. See, e.g., http://www.ncbi.nlm.nih.gov. Another non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

The invention also provides TOP chimeric or fusion proteins. As used herein, a TOP “chimeric protein” or “fusion protein” comprises a TOP polypeptide operatively linked to a non-TOP polypeptide. A “TOP polypeptide” refers to a polypeptide having an amino acid sequence corresponding to TOP, whereas a “non-TOP polypeptide” refers to a polypeptide having an amino acid sequence corresponding to a protein which is not substantially identical to the TOP protein, e.g., a protein which is different from the TOP protein and which is derived from the same or a different organism. Within a TOP fusion protein the TOP polypeptide can correspond to all or a portion of a TOP protein, preferably at least one biologically active portion of a TOP protein. Within the fusion protein, the term “operatively linked” is intended to indicate that the TOP polypeptide and the non-TOP polypeptide are fused in-frame to each other. The non-TOP polypeptide can be fused to the N-terminus or C-terminus of the TOP polypeptide.

One useful fusion protein is a GST-TOP fusion protein in which the TOP sequences are fused to the C-terminus of the GST sequences. Such fusion proteins can facilitate the purification of recombinant TOP.

In another embodiment, the fusion protein is a TOP protein containing a heterologous signal sequence at its N-terminus. For example, the native TOP signal sequence can be removed and replaced with a signal sequence from another protein. In certain host cells (e.g., mammalian host cells), expression and/or secretion of TOP can be increased through use of a heterologous signal sequence. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Molecular Cloning, Sambrook et al, second edition, Cold Spring Harbor Laboratory Press, 1989) and the protein A secretory signal (Pharmacia Biotech; Piscataway, N.J.).

In yet another embodiment, the fusion protein is a TOP-immunoglobulin fusion protein in which all or part of TOP is fused to sequences derived from a member of the immunoglobulin protein family. The TOP-immunoglobulin fusion proteins of the invention can be used as immunogens to produce TOP antibodies in a subject, to purify TOP ligands and in screening assays to identify molecules that inhibit the interaction of TOP with an antigenic peptide.

In general, a TOP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al. eds., John Wiley & Sons: 1992). Moreover, many expression vectors are commercially available that already encode a fusion moiety (e.g., a GST polypeptide). A TOP-encoding nucleic acid can be cloned into such an expression vector such that the fusion moiety is linked in-frame to the TOP protein.

The present invention also pertains to variants of the TOP proteins which function as either TOP agonists (mimetics) or as TOP antagonists. Variants of the TOP protein can be generated by mutagenesis, e.g., discrete point mutation or truncation of the TOP protein. An agonist of the TOP protein can retain substantially the same, or a subset, of the biological activities of the naturally occurring form of the TOP protein. An antagonist of the TOP protein can inhibit one or more of the activities of the naturally occurring form of the TOP protein by, for example, competitively binding to a downstream or upstream member of a cellular signaling cascade which includes the TOP protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the TOP proteins.

Variants of the TOP protein which function as either TOP agonists (mimetics) or as TOP antagonists can be identified by screening combinatorial libraries of mutants, e.g., truncation mutants, of the TOP protein for TOP protein agonist or antagonist activity. In one embodiment, a variegated library of TOP variants is generated by combinatorial mutagenesis at the nucleic acid level and is encoded by a variegated gene library. A variegated library of TOP variants can be produced by, for example, enzymatically ligating a mixture of synthetic oligonucleotides into gene sequences such that a degenerate set of potential TOP sequences is expressible as individual polypeptides, or alternatively, as a set of larger fusion proteins (e.g., for phage display) containing the set of TOP sequences therein. There are a variety of methods which can be used to produce libraries of potential TOP variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TOP sequences. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983, Nucleic Acid Res. 11:477).

In addition, libraries of fragments of the TOP protein coding sequence can be used to generate a variegated population of TOP fragments for screening and subsequent selection of variants of a TOP protein. In one embodiment, a library of coding sequence fragments can be generated by treating a double-stranded PCR fragment of a TOP coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with SI nuclease, and ligating the resulting fragment library into an expression vector. By this method, an expression library can be derived which encodes N-terminal and internal fragments of various sizes of the TOP protein. Such fragments are useful, e.g., for generating antibodies that specifically bind to particular TOP epitopes.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TOP proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify TOP variants (Arkin and Yourvan, 1992, Proc. Natl. Acad. Sci. USA 89:7811-7815; Delgrave et al., 1993, Protein Engineering 6(3):327-331). Mutant TOP proteins can be useful, for example, for enhancing the immunosuppressive effect of TOP overexpression. In this case, a mutant TOP protein may have even greater immunosuppressive activity when expressed in a cell compared to over-expression of wild-type TOP.

An isolated TOP protein, or a portion or fragment thereof, can be used as an immunogen to generate antibodies that bind TOP using standard techniques for polyclonal and monoclonal antibody preparation. The full-length TOP protein can be used or, alternatively, the invention provides antigenic peptide fragments of TOP for use as immunogens. The antigenic peptide of TOP comprises at least 8 (generally 10, 15, 20, or 30) amino acid residues of the amino acid sequence shown in SEQ ID NO:2 and encompasses an epitope of TOP such that an antibody raised against the peptide forms a specific immune complex with TOP.

Preferred epitopes encompassed by the antigenic peptide are regions of TOP that are located on the surface of the protein, e.g., hydrophilic regions. Hydrophilic regions and antigenic regions can be identified using standard analytical tools well-known to those skilled in the art.

A TOP immunogen typically is used to prepare antibodies by immunizing a suitable subject, (e.g., rabbit, goat, mouse, or other mammal) with the immunogen. An appropriate immunogenic preparation can contain, for example, recombinantly expressed TOP protein or a chemically synthesized TOP polypeptide. The preparation can further include an adjuvant, such as Freund's complete or incomplete adjuvant, or similar immunostimulatory agent.

Immunization of a suitable subject with an immunogenic TOP preparation induces a polyclonal anti-TOP antibody response. Accordingly, another aspect of the invention pertains to anti-TOP antibodies. The term “antibody” as used herein refers to immunoglobulin molecules and immunologically active portions of immunoglobulin molecules, i.e., molecules that contain an antigen-binding site which specifically binds an antigen, such as TOP. A molecule that “specifically binds” to TOP is a molecule that binds to TOP, but does not substantially bind other molecules in a sample, e.g., a biological sample, which naturally contains TOP. Examples of immunologically active portions of immunoglobulin molecules include F(ab) and F(ab′)2 fragments which can be generated by treating the antibody with an enzyme such as pepsin.

The invention provides polyclonal and monoclonal antibodies that bind TOP. The term “monoclonal antibody” or “monoclonal antibody composition”, as used herein, refers to a population of antibody molecules that contain only one species of an antigen binding site capable of immunoreacting with a particular epitope of TOP. A monoclonal antibody composition thus typically displays a single binding affinity for a particular TOP protein with which it immunoreacts.

Polyclonal anti-TOP antibodies can be prepared as described above by immunizing a suitable subject with a TOP immunogen. The anti-TOP antibody titer in the immunized subject can be monitored over time by standard techniques, such as with an enzyme linked immunosorbent assay (ELISA) using immobilized TOP. If desired, the antibody molecules directed against TOP can be isolated from the mammal (e.g., from the blood) and further purified by well-known techniques, such as protein A chromatography to obtain the IgG fraction. At an appropriate time after immunization, e.g., when the anti-TOP antibody titers are highest, antibody-producing cells can be obtained from the subject and used to prepare monoclonal antibodies by standard techniques, such as the hybridoma technique originally described by Kohler and Milstein, 1975, Nature 256:495-497), the human B cell hybridoma technique (Kozbor et al., 1983, Immunol Today 4:72), the EBV-hybridoma technique (Cole et al., 1985, Monoclonal Antibodies and Cancer Therapy, Alan R. Liss, Inc., pp. 77-96) or trioma techniques. The technology for producing various antibodies monoclonal antibody hybridomas is well known (see generally Current Protocols in Immunology, 1994, Coligan et al. (eds.) John Wiley & Sons, Inc., New York, N.Y.). Briefly, an immortal cell line (typically a myeloma) is fused to lymphocytes (typically splenocytes) from a mammal immunized with a TOP immunogen as described above, and the culture supernatants of the resulting hybridoma cells are screened to identify a hybridoma producing a monoclonal antibody that binds TOP.

Any of the many well known protocols used for fusing lymphocytes and immortalized cell lines can be applied for the purpose of generating an anti-TOP monoclonal antibody (see, e.g., Current Protocols in Immunology, supra; Galfre et al., 1977, Nature 266:55052; Kenneth, in Monoclonal Antibodies: A New Dimension In Biological Analyses, Plenum Publishing Corp., New York, N.Y. (1980); and Lemer, 1981, Yale J. Biol. Med. 54:387-402). Moreover, the ordinarily skilled worker will appreciate that there are many variations of such methods that also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind TOP, e.g., using a standard ELISA assay.

Alternative to preparing monoclonal antibody-secreting hybridomas, a monoclonal anti-TOP antibody can be identified and isolated by screening a recombinant combinatorial immunoglobulin library (e.g., an antibody phage display library) with TOP to thereby isolate immunoglobulin library members that bind TOP. Kits for generating and screening phage display libraries are commercially available (e.g., the Pharmacia Recombinant Phage Antibody System, Catalog No. 27-9400-01; and the Stratagene SurfZAPO Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-TOP antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-TOP antibody (e.g., monoclonal antibody) can be used to isolate TOP by standard techniques, such as affinity chromatography or immunoprecipitation. An anti-TOP antibody can facilitate the purification of natural TOP from cells and of recombinantly produced TOP expressed in host cells. Moreover, an anti-TOP antibody can be used to detect TOP protein (e.g., in a cellular lysate or cell supernatant) in order to evaluate the abundance and pattern of expression of the TOP protein. Anti-TOP antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

Completely human antibodies can also be produced, for example, by using transgenic mice that are incapable of expressing endogenous immunoglobulin heavy and light chains genes, but that can express human heavy and light chain genes. The transgenic mice are immunized in the normal fashion with a selected antigen. Monoclonal antibodies directed against the antigen can be obtain using conventional hybridoma technology. The human immunoglobulin transgenes of harbored by the transgenic mice rearrange during B cell differentiation, subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, human antibodies directed against a selected antigen can be provided by Abgenix, Inc. (Fremont, Calif.) and GenPharm, Inc. (Palo Alto, Calif.).

Expression Vectors

Another aspect of the invention pertains to vectors, preferably expression vectors, containing a nucleic acid encoding TOP (or a portion thereof). These vectors can be used to transfect cells with the TOP gene to enable the cells to express, or overexpress TOP, either in vitro or in vivo. For example, these vectors can be used to produce cells that will express and create large amounts of TOP that can be isolated from the culture medium once the cells have been disrupted. Alternatively, the vectors can be used to transfect cells that are to be introduced or implanted into a host, such as an animal or human patient in need of the cells. The cells, can be for example, stem cells or bone marrow stromal cells, e.g., stem cells that have been engineered to include a desired gene that is to be expressed in the host. To reduce the immunogenic effect of these cells, they can also be engineered to include the TOP gene, to overexpress TOP and thereby ensure that fewer, or no, antigenic peptides appear on the surface of the engineered cells once implanted into the patient.

As used herein, the term “vector” refers to a nucleic acid molecule capable of transporting another nucleic acid to which it has been linked. One type of vector is a “plasmid,” which refers to a circular, double-stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operatively linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which are operatively linked to the nucleic acid sequence to be expressed. Within a recombinant expression vector, “operably linked” is intended to mean that the nucleotide sequence of interest is linked to one or more sequences in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., TOP proteins, mutant forms of TOP, fusion proteins, etc.).

The recombinant expression vectors of the invention can be designed for expression of TOP in prokaryotic or eukaryotic cells, e.g., bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase. Such vectors are used to express TOP or PSA proteins or polypeptides, e.g., for producing antibodies or for expression in host cells for modulation of MHC class I antigen presentation.

Expression of proteins in prokaryotes is generally carried out in E. coli (although other suitable prokaryotes known in the art can be used) with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident Xprophage harboring a T7 gn1 gene under the transcriptional control of the lacUV 5 promoter.

One strategy to maximize recombinant protein expression in E. coli is to express the protein in host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the TOP expression vector is a yeast expression vector. Examples of vectors for expression in yeast S. cerivisae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and picZ (InVitrogen Corp, San Diego, Calif.).

Alternatively, TOP can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells, such as stem cells or bone marrow stromal cells, using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions can be provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al. (supra). Other suitable expression systems are known in the art.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the OL-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to TOP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

The invention also includes a cell in which TOP or PSA expression is modulated by altering the expression of an endogenous TOP or PSA gene. Such methods are known in the art, for example, see U.S. Pat. Nos. 5, 968,502; 6,270,989, and 6,361,972. In one such method, targeted homologous recombination can be used to increase TOP expression by altering a regulatory sequence of an endogenous TOP gene. This process can be carried out in vitro and those cells in which TOP has been modulated can be transplanted into an animal in need of treatment with such cells, e.g., for immunosupprssion.

Another aspect of the invention pertains to host cells into which a recombinant expression vector of the invention has been introduced. The terms “host cell,” “recombinant host cell,” and “recombinant cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, TOP protein can be expressed in bacterial cells such as E. coli, insect cells, yeast, or mammalian cells (such as stem cells or bone marrow stromal cells, or cells to be cultured, such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. To identify and select these integrants, a gene that encodes a selectable marker (e.g., resistance to antibiotics) is generally introduced into the host cells along with the gene of interest. Selectable markers include those which confer resistance to drugs, such as G418, hygromycin and methotrexate. Nucleic acid encoding a selectable marker can be introduced into a host cell on the same vector as that encoding TOP or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) TOP protein. This includes host cells that overexpress TOP compared to a wild-type cell. Accordingly, the invention further provides methods for producing TOP protein using the host cells of the invention. In one embodiment, the method comprises culturing the host cell of invention (into which a recombinant expression vector encoding TOP has been introduced) in a suitable medium such that TOP protein is produced. In another embodiment, the method further comprises isolating TOP from the medium or the host cell.

The TOP nucleic acid molecules, TOP proteins, and anti-TOP antibodies, and inhibitors and activators of TOP expression or activity (also referred to herein as “active compounds”) can be incorporated into pharmaceutical compositions suitable for administration.

Therapeutic compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In some cases, a vector of the invention or a modulator of TOP or PSA is administered to a subject by injection. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fingi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a TOP encoding nucleic acid, TOP proteins or peptides, or anti-TOP antibodies) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation include vacuum drying or freeze-drying which yields a powder of the active ingredient plus any additional desired ingredient from a previously sterile-filtered solution thereof.

DNA vaccines can be administered orally, and as such, may also include compositions that modulate TOP and/or PSA expression or activity. Oral compositions can also contain other modulators of TOP and/or PSA expression or activity as described herein. Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

Vaccines

Vaccines can be administered in any pharmaceutically acceptable carrier or diluent, including water, normal saline, phosphate buffered saline, or a solution of bicarbonate such as 0.1 M NaHCO3. The carrier or diluent is selected on the basis of the mode and route of administration, and standard pharmaceutical practice. Additional suitable pharmaceutical carriers and diluents, as well as pharmaceutical necessities for their use in pharmaceutical formulations, are described, e.g., in Remington's Pharmaceutical Sciences, a standard reference text in this field.

The amount of vaccine administered will depend on the particular vaccine antigen, whether an adjuvant is co-administered, the mode and frequency of administration, and the desired effect. Each of these considerations are understood by skilled artisans. In general, the vaccine antigen is administered in amounts ranging between, e.g., 1 μg and 100 mg. If adjuvants, such as the cells described herein having inhibited TOP activity and/or expression, are administered with the vaccines, amounts ranging from between e.g., 1 ng and 1 mg of antigen can be used. The dosage can also be calculated empirically, e.g., based on animal studies and, expressed in terms of a patient's weight, can range from 0.1 to 500 μg/kg.

Skilled artisans will recognize that the vaccine described herein can be administered in conjunction with other methods of treatment. For example, the vaccine can be administered before, during, or after administration of chemotherapeutic agents, radiation therapy, or surgical ablation of a malignant tumor or benign growth of cells.

A number of adjuvants, in addition to those described above, are known to skilled artisans and may be used to perform the immunization described herein. For example, cholera toxin (CT), the heat-labile enterotoxin of Escherichia coli (LT), or fragments or derivatives thereof having adjuvant activity, can be used for transmucosal administration. Alternatively, adjuvants such as RIBI (ImmunoChem, Hamilton, VT) or aluminum hydroxide can be used for parenteral administration.

Fusion proteins containing nucleosomes fused to an adjuvant (e.g., CT, LT, or a fragment or derivative thereof having adjuvant activity), are considered within the scope of the invention, and can be prepared using standard methods (see, e.g., Ausubel et al.). In addition, the vaccines of the invention can be covalently coupled or cross-linked to adjuvants. Methods of covalently coupling or chemically cross-linking adjuvants to antigens are described in, e.g., Cryz et al. (Vaccine, 13:67-71, 1994), Liang et al. (J. Immunol., 141:1495-1501, 1988), and Czerkinsky et al. (Infection and Immunity, 57:1072-1077, 1989).

The cells described herein can be administered as a physiologically acceptable formulation containing an excipient. Examples of excipients which may be included with the formulation are buffers such as citrate buffer, phosphate buffer, acetate buffer, and bicarbonate buffer, amino acids, urea, alcohols, ascorbic acid, proteins, such as serum albumin and gelatin, EDTA, sodium chloride, polyvinylpyrollidone, mannitol, sorbitol, glycerol, propylene glycol, and polyethylene glycol (e.g., PEG-4000, PEG-6000).

The transfected cells described herein can be used as part of a tissue engineering construct. The cells in these constructs have modulated immunoresponse characteristics (i.e., decreased antigenic presentation) such that the exogenous tissue construct that results is less susceptible to a deleterious autoimmune response from the host, and subsequent rejection of the new tissue. The constructs can comprise an organic or polymeric support, preferably of a biodegradable nature.

The methods of identifying peptides (e.g., antigens resistant to TOP degradation) and methods of identifying compounds that are immunomodulators utilize compositions mentioned herein, as well as other reagents and assay methodologies to assess the character of the peptides and compounds of interest. The following description provides further detail on the manner of making and using the invention and its various embodiments.

Screening Assays

The invention provides methods (also referred to herein as a “screening assay”) for identifying modulators, i.e., candidate or test compounds or agents (e.g., peptides, peptidomimetics, carbohydrates, aptamers, oligosaccharides, nucleic acids such as antisense strands or ribozymes, organic or inorganic small molecules, or other chemicals or drugs) that bind to a TOP protein and/or have a stimulatory or inhibitory effect on, for example, TOP expression or activity (e.g., by binding to nucleic acids that encode TOP).

The invention provides assays for screening test compounds that bind to or modulate the activity of the TOP protein or polypeptide or biologically active portion thereof. Other embodiments entail the use of a soluble form of TOP. Test compounds can also be screened for their ability to inhibit expression of TOP, by binding to or otherwise interfering with the expression of the TOP gene. Test compounds that bind to TOP are candidate immunomodulators, that can be further tested to confirm their ability to modulate, either increase or decrease, TOP activity. Test compounds that bind to the TOP gene are also candidate immunomodulators, that can be further tested to confirm their ability to modulate, either increase or decrease, the expression of TOP by nucleic acids that encode TOP.

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; and Felici (1991) J. Mol. Biol. 222:301-310).

The invention includes assays employing soluble TOP. Such assays entail contacting a TOP protein or biologically active portion thereof with a test compound and determining the ability of the test compound to bind to TOP protein or biologically active portion thereof. Binding of the test compound to TOP protein can be determined either directly or indirectly using the approaches described herein. In one embodiment, the assay includes contacting TOP protein or biologically active portion thereof with a known compound which binds TOP to form an assay mixture, contacting the assay mixture with a test compound, and determining the ability of the test compound to interact with TOP protein and displace the known compound or prevent binding of TOP to the known compound.

In another embodiment, an assay is a cell-free assay comprising contacting TOP protein or biologically active portion thereof with a test compound and determining the ability of the test compound to modulate (e.g., stimulate or inhibit) the activity of TOP or a biologically active portion thereof. Determining the ability of the test compound to modulate the activity of TOP can be accomplished, for example, by determining the ability of TOP to bind to a test compound by one of the methods described herein for determining direct binding. In an alternative embodiment, determining the ability of the test compound to modulate the activity of TOP can be accomplished by determining the ability of the agent to alter the activity of a TOP target molecule. For example, the catalytic/enzymatic activity of the target molecule on an appropriate substrate can be determined.

In yet another embodiment, the cell-free assay comprises contacting the TOP protein or biologically active portion thereof with a known compound which binds TOP to form an assay mixture, contacting the assay mixture with a test compound, and determining the ability of the test compound to interact with a TOP protein, wherein determining the ability of the test compound to interact with a TOP protein comprises determining the ability of the TOP protein to preferentially bind to or modulate the activity of a TOP target molecule.

Determining the ability of test compounds to bind to the TOP protein can be accomplished, for example, by coupling the test compounds with a radioisotope or enzymatic label such that binding of the test compound to the TOP protein or biologically active portion thereof can be determined by detecting the labeled compound in a complex. For example, test compounds can be labeled with ¹²⁵I, ³⁵S,¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of TOP protein, or a biologically active portion thereof, on the cell surface with a known compound which binds TOP to form an assay mixture, contacting the assay mixture with a test compound, and determining the ability of the test compound to interact with a TOP protein, wherein determining the ability of the test compound to interact with a TOP protein comprises determining the ability of the test compound to preferentially bind to TOP or a biologically active portion thereof as compared to the known compound.

In more than one embodiment of the above assay methods of the present invention, it may be desirable to immobilize either TOP or the corresponding target molecule to facilitate separation of complexed from uncomplexed forms of one or both of the proteins, as well as to accommodate automation of the assay. Binding of a test compound to TOP or interaction of TOP with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/fusion proteins or glutathione-S-transferase/target fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or TOP protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of TOP binding or activity determined using standard techniques.

Other techniques for immobilizing proteins on matrices can also be used in the screening assays of the invention. For example, either TOP or the corresponding target molecule can be immobilized utilizing conjugation of biotin and streptavidin. Biotinylated TOP or the corresponding target molecule can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with TOP or the corresponding target molecule but which do not interfere with binding of the TOP protein to its target molecule can be derivatized to the wells of the plate, and unbound target or TOP trapped in the wells by antibody conjugation. Methods for detecting such complexes, in addition to those described above for the GST-immobilized complexes, include immunodetection of complexes using antibodies reactive with the TOP or corresponding target molecule, as well as enzyme-linked assays which rely on detecting an enzymatic activity associated with the TOP or corresponding target molecule.

In another embodiment, modulators of TOP expression are identified in a cell-based assay in which a cell is contacted with a candidate compound and the expression of TOP mRNA or protein in the cell is determined. The level of expression of TOP mRNA or protein in the presence of the candidate compound is compared to the level of expression of TOP mRNA or protein in the absence of the candidate compound. The candidate compound can then be identified as a modulator of TOP expression based on this comparison. For example, when expression of TOP mRNA or protein is greater (statistically significantly greater) in the presence of the candidate compound than in its absence, the candidate compound is identified as a stimulator of TOP mRNA or protein expression. Alternatively, when expression of TOP mRNA or protein is less (statistically significantly less) in the presence of the candidate compound than in its absence, the candidate compound is identified as an inhibitor of TOP mRNA or protein expression. The level of TOP mRNA or protein expression in the cells can be determined by methods described herein for detecting TOP mRNA or protein.

For example, when activity of TOP is greater (statistically significantly greater) in the presence of the candidate compound than in its absence, the candidate compound is identified as a stimulator of TOP mRNA or protein expression. Alternatively, when the activity of TOP is less (statistically significantly less) in the presence of the candidate compound than in its absence, the candidate compound is identified as an inhibitor of TOP activity.

In yet another aspect of the invention, TOP protein can be used a “bait protein” in a two-hybrid assay or three-hybrid assay (see, e.g., U.S. Pat. No. 5,283,317; Zervos et al., 1993, Cell 72:223-232; Madura et al., 1993, J. Biol. Chem. 268:12046-12054; Bartel et al., 1993, Bio/Techniques 14:920-924; Iwabuchi et al., 1993, Oncogene 8:1693-1696; and PCT Publication No. WO 94/10300), to identify other proteins, which bind to or interact with TOP and modulate activity.

The two-hybrid system is based on the modular nature of most transcription factors, which consist of separable DNA-binding and activation domains. Briefly, the assay utilizes two different DNA constructs. In one construct, the gene that codes for the protein of interest, e.g., TOP, is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with TOP.

### EXAMPLES

The invention is further described in the following examples, which do not limit the scope of the invention described in the claims.

### Example 1

Antigenic Peptides

A variety of known antigenic peptides, whose presentation has been extensively studied in vivo, were examined in the experiments described herein. Peptides were synthesized by Macromolecular Resources (Colorado State University, Fort Collins, CO) or the Dana Farber Cancer Research Institute core facility (Boston, Mass.) and were at least 90% pure by HPLC analysis. The peptides were dissolved at 10 mg/ml in DMSO and stored at 80° C. The internally quenched fluorogenic substrate Mcc-Pro-Leu-Gly-Pro-D-Lys(Dnp)-OH (Mcc-PLGPK-Dnp; SEQ ID NO:3) and the inhibitor Cpp-AAF-pAb were obtained from Calbiochem-Novabiochem (San Diego, Calif.). The fluorogenic substrate for prolyl oligopeptidase (Z-GP-Amc), tripeptidyl peptidase II (AAF-Amc) and aminopeptidases (A-Amc, L-Amc) were obtained from Bachem (Basel, Switzerland). Bestatin, o-phenanthroline, PMSF and E64 were purchased from Sigma (St. Louis, Mo.). The following six antigenic peptides were studied: ASNENMETM (SEQ ID NO:4; influenzavirus nucleoprotein, Flu NP, residues 366-374), SIINFEKL (SEQ ID NO:5; ovalbumin, residues 257-264 of Genbank accession no. V00383), TYQRTRALV (SEQ ID NO:6; Flu NP, residues 147-154 of Genbank accession no. M38279), RGPGRAFVTI (SEQ ID NO:7; HIV gp160, residues 318-327 of Genbank accession no. AF116045), TPHPARIGL (SEQ ID NO:8; β-galactosidase, residues 876-884), and FAPGNYPAL (SEQ ID NO:10; Sendai Virus NP, residues 324-332 Genbank accession nos. X17218 and X51331). All of these peptides had previously been found associated with murine MHC class-I-alleles and shown to induce a T cell immune response (Rammensee et al., 1995, Immunogenetics, 41(4):178-228); consequently, they have often been used in studies of antigen presentation.

Recombinant human interferon-gamma was obtained from Biogen, Inc. (Cambridge, Mass.). The proteasome inhibitor MG132 (Cbz-Leu-Leu-leucinal) was obtained from ProScript Inc. (Cambridge, Mass.), and phosphinic peptide inhibitor of TOP, Z-Pheψ(PO₂CH₂)Ala-Arg-Phe-OH (SEQ ID NO:9). Rabbit antiserum against purified bovine leucine aminopeptidase was generated in this laboratory. Monoclonal antibody against proteasomal a-subunit HC3 was purchased from Affinity Research Products, Ltd. (Mamhead, UK).

Cell Lines

The human cervical carcinoma cell line HeLa S3 used in the experiments described herein was obtained from the American Type Culture Collection (ATCC) and grown in DMEM (Irving Scientific, Santa Ana, Calif.) supplemented with 10% fetal calf serum and antibiotics. HeLa S3 cells were grown in 100 mm dishes containing 10 ml of culture medium and were treated for different periods of time with 150 U/ml of IFN-γ (type 2 interferon) as detailed below.

Cell Extracts

To prepare cell extracts used in the experiments described herein, cells from confluent cultures were washed twice with ice-cold PBS, pH 7.4, and were removed into a homogenization buffer using a cell lifter. Cells were homogenized in a Dounce homogenizer and by vortexing with glass beads in 50 mM Tris-HCl, 5 mM MgCl₂, 2 mM ATP, 1 mM dithiothreitol (DTT) and 250 mM sucrose, pH 7.4. Cytosolic extracts were prepared by centrifugation of the homogenates for ten minutes at 20,000×g and for one hour at 100,000×g. Most of the proteasomes were removed by an additional six hours centrifugation at 100,000×g. All extracts were stored at −80° C. until use. The residual proteasomal activity in the extracts was inhibited by pre-incubation with 20 μM MG132 for 15 minutes at room temperature. Protein concentration in the extracts was determined with the Coomasie Plus Protein Assay Reagent (Pierce).

Fractionation of 0.5 mg of HeLa cell extract (6 hour/100,000×g supernatant) was performed by ion exchange chromatography on a 1 ml MonoQ 5/5 column (Pharmacia, Upsala, Sweden) in 50 mM Tris-HCl buffer, 5 mM β-mercaptoethanol and 0.05% Brij 35 pH 7.8. Bound proteins were eluted with a 20 minute linear gradient from 0 to 0.5 M sodium chloride and with a flow rate of 1 ml/minute. The protein elution profiles were measured at 280 nm, and fractions of 0.5 ml were collected for further analysis.

Peptidase Assays

Peptidase activities were assayed in a continuous assay at 37° C. with 10 mg of cytosolic extract from HeLa cells and the specific fluorogenic substrate in 500 ml volume of 50 mM Hepes-KOH, pH 7.6, 2 mM MgCl2, 0.1 mM DTT. Thimet oligopeptidase activity was analyzed using the quenched fluorogenic substrates Mcc-PLGPK-Dnp at 20 μM, and the fluorescent product was measured at an excitation wavelength of 345 nm and an emission wavelength of 405 nm in a SLM AMINCO luminescence spectrometer. The activity of prolyl oligopeptidase (POP) was measured with Z-GP-Amc at 100 μM final concentration. The release of Amc was monitored at excitation/emission wavelengths of 380/460 nm. The tripeptidyl peptidase II (TPP-II) was assayed with 100 μM AAF-Amc in the presence of aminopeptidase inhibitor bestatin. The activity of cytosolic aminopeptidases was determined routinely with 100 μM A-Amc or L-Amc, and the levels of the insulin-degrading enzyme were measured by immunoblotting.

Peptidase assays are useful for assessing whether a cell comprising an engineered TOP nucleic acid sequence is expressing a desired amount of TOP. For example, whether a cell has increased TOP activity compared a control cell (such as an ancestor of the cell, the ancestor not being engineered). Such assays can also be used to asses whether an engineered cell has a decreased amount of TOP activity and thus is useful, e.g., for use in a vaccine composition.

HPLC Analysis of Peptide Metabolism

Five nmol of the synthetic peptides were incubated at 37° C. for various time intervals with 10 mg HeLa extract in 100 ml 50 mM Hepes-KOH, pH 7.6, 2 mM MgCl₂, 0.1 mM DTT, 20 μM MG132. The reaction was terminated by addition of 100 ml 20% trichloracetic acid followed by a 15 minute incubation on ice and removal of the precipitated protein by centrifugation for 15 minutes at 20,000×g. The peptide-containing supernatant was subjected to reversed-phase HPLC on a 4.6×250 mm Vidac 5 mm C18-column (Vidac, Hesperia, Calif.) in 0.06% trifluoracetic acid and with a flow rate of 1 ml/minute. Elution was performed with a 30 minute linear gradient from 5 to 60% acetonitrile. The eluting peptides were calculated by integration of peptide peaks on chomatograms and are given in arbitrary units (Beninga et al., 1998, J Biol. Chem., 273(30):18734-18742). To study the effects of inhibitors, the extracts were preincubated with the indicated inhibitors for 15-30 minutes at room temperature.

Such methods can be used in screening assays to identify inhibitors of TOP or PSA.

Immunoblot Analysis

The identification and quantification of thimet oligopeptidase in soluble extracts was performed by separation of 50 mg of crude extract or 40 μl of each sample from fractionated extracts on a 10% SDS-polyacrylamide gel followed by transfer of the proteins to an hnmobilon P membrane (Millipore). The filters were blocked for 30 minutes at room temperature with 0.5% milk powder in phosphate buffered saline (PBS) and incubated overnight at 4° C. with the specific primary antibodies or antiserum. Bound antibodies on the immunoblots were detected with the alkaline phosphatase (AP) or horseradish peroxidase (HRP) conjugated secondary antibodies. Signals were developed using ECL (Amersham Pharmacia Biotech, Little Chalfont, UK) with the AP-substrate, CDP-Star (Tropix, Bedford, Mass.) or HRP-substrate.

Effect of in Vivo Overexpression of TOP on Antigenic Peptide Presentation

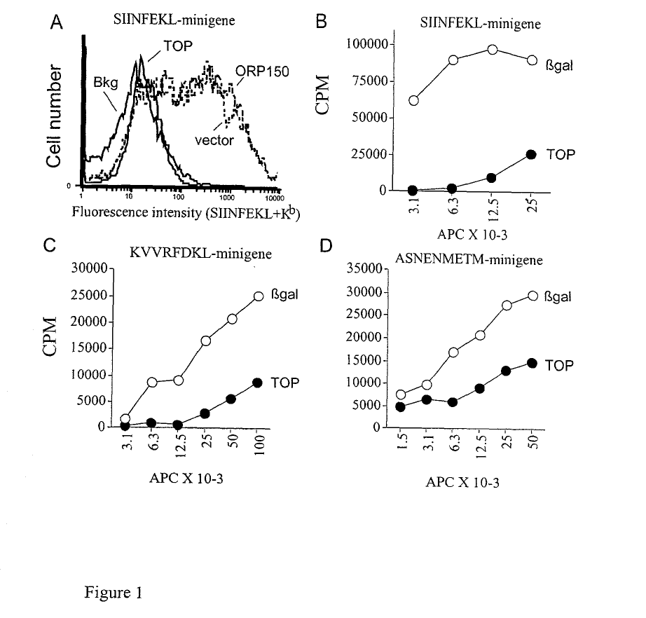

To test whether TOP can influence antigen presentation, the effects of transiently overexpressing TOP in vivo on the presentation of SIINFEKL (SEQ ID NO:5) peptide produced in the cytoplasm from a synthetic minigene were studied. In one set of experiments, expression vectors encoding TOP and SIINFEKL were transiently co-transfected into Kbexpressing COS7 cells. The presentation of SIINFEKL on MHC class I Kb molecules was detected by staining with a SIlNFEKL+Kb-specific monoclonal antibody (25D1) (Porgador et al., 1997, Immunity. 6:715-726) and quantified by flow cytometry. Briefly, Kb-Cos7 cells were co-transfected with a SIINFEKL minigene in pCDNA3 and a pTracer plasmid encoding green fluorescence protein (GFP), GFP+human TOP or GFP+ORP 150 (a control protein) using FuGENE™ (Roche Applied Science). After 48 hours, the cells were stained with an anti-SIINFEKL+Kb specific nAb (25D1) (Porgador et al., 1997, supra) followed by pbycoerythrin-conjugated anti-immunoglobulin antibody and fluorescence was measured by flow fluorocytometry. The histograms of FIG. 1A display 25D1 staining intensity versus cell number for pTracer-transfected (GFP-positive) cells. The generation of SIINFEKL-Kb complexes was markedly reduced in cells expressing TOP as compared to cells expressing control proteins, green fluorescence protein (GFP) or ORP150 (FIG. 1A).

Such methods are useful for assessing whether cells are suitable for use in protocols where reduced immunogenicity is desired.

In another set of experiments TOP and T7 polymerase were expressed from vaccinia recombinants in Kb-expressing E36 cells which were then transfected with a SIINFEKL minigene under the control of a T7 promoter. 1.25 hours after transfection, the antigen presenting cells were fixed, and the presence of SIINFEKL on Kb molecules was assayed by measuring the production of IL-2 from a specific T cell hybridoma. Briefly, in these experiments, Kb-E36 antigen presenting cells were infected for two hours with vaccinia-T7 polymerase and either vaccinia-TOP or vaccinia-Bgal and then transfected with a SIINFEKL minigene under the control of a T7 promoter in a pBluescript plasmid (1 μg) using Lipofectin liposomes (Life Technologies) as described (Craiu, et al., 1997, Proc. Natl. Acad. Sci. USA. 94, 10850-55). After 1.25 hours the cells were fixed with 1% paraformaldehyde and the indicated number (see FIG. 1) incubated with the SIINFEKL+Kb specific T cell hybrid, RF33.70 (Craim et al., 1997, Proc. Natl. Acad. Sci. USA. 94:10850-10855). After 18 hours the production of IL-2 from RF33.70 was measured using CTLL2 cells as described (Craiu, et al. 1997, Proc. Natl. Acad. Sci. USA. 94:10850-10855). Again the presentation of SIINFEKL on Kb was markedly inhibited in cells overexpressing TOP compared to cells infected with a vaccinia control (lacZ) (FIG. 1B).

TOP also inhibited the presentation on Kb of another ovalbumin peptide, KVVRFDKL (SEQ ID NO: 11; FIG. 1C). This was shown using methods similar to those described supra (as for FIG. 1B) except that a KVVRFDKL minigene was used instead of SIINFEKL, the 1G8 hybridoma (Cole et al. IntImmunol 6:1767-1775 (1994)); gift from J. McCluskey, Flinders Medical Center) was used instead of RF33.70 and the transfection time was 2.5 hours.

Using similar vaccinia expression systems and T cell hybridoma assays, the question of whether TOP would affect the presentation of other antigenic peptides expressed in the cytoplasm was investigated. In related studies, we had previously found that TOP is also the primary peptidase in cell extracts that degrades the influenza-derived peptide ASNENMETM (SEQ ID NO:4). These results were confirmed using yet another antigen. The experiment was similar to that shown in FIG. 1B except Db-E36 cells were used instead of Kb-E36, vaccinia-T7 polymerase was omitted and after 1.5 hours the cells were infected for 1.5 hours with a vaccinia recombinant expressing ASNENMETM (SEQ ID NO:4) instead of being transfected with a SIINFEKL minigene and the 12.3 hybridoma (Deckhut et al., 1993, J. Imunol. 151:2658-2666; from D. Woodland, Trudeau Institute, NY) was used instead of RF33.70. Overexpression of TOP inhibited the presentation on this influenza epitope on Db class I molecules (See FIG. 1D).

A large increase in TOP content in the transfected cells was confirmed by Western blot. Western blots confirmed that TOP levels were markedly increased in cells infected with the vaccinia-TOP recombinant. A similar reduction in antigen presentation was observed upon vaccinia expression of TOP in L cells. In these experiments, the TOP-overexpressing cells showed no reduction in viability, as judged by exclusion of trypan blue, or synthesis of proteins, as assessed by incorporation of 35S-met into proteins. Therefore, TOP can limit the presentation of SIINFEKL expressed in the cytoplasm from a minigene without causing apparent toxicity.

Thus TOP can reduce MHC class I presentation of at least three antigenic peptides expressed from minigenes and in several different types of cells.

### Example 2

Effect of in Vivo Overexpression of TOP on Antigenic Peptide (of Full-Length Origin) Presentation

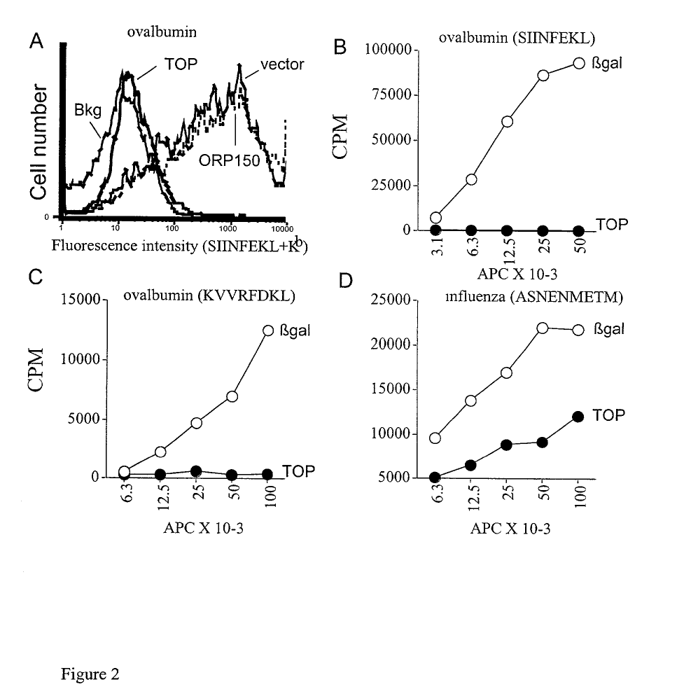

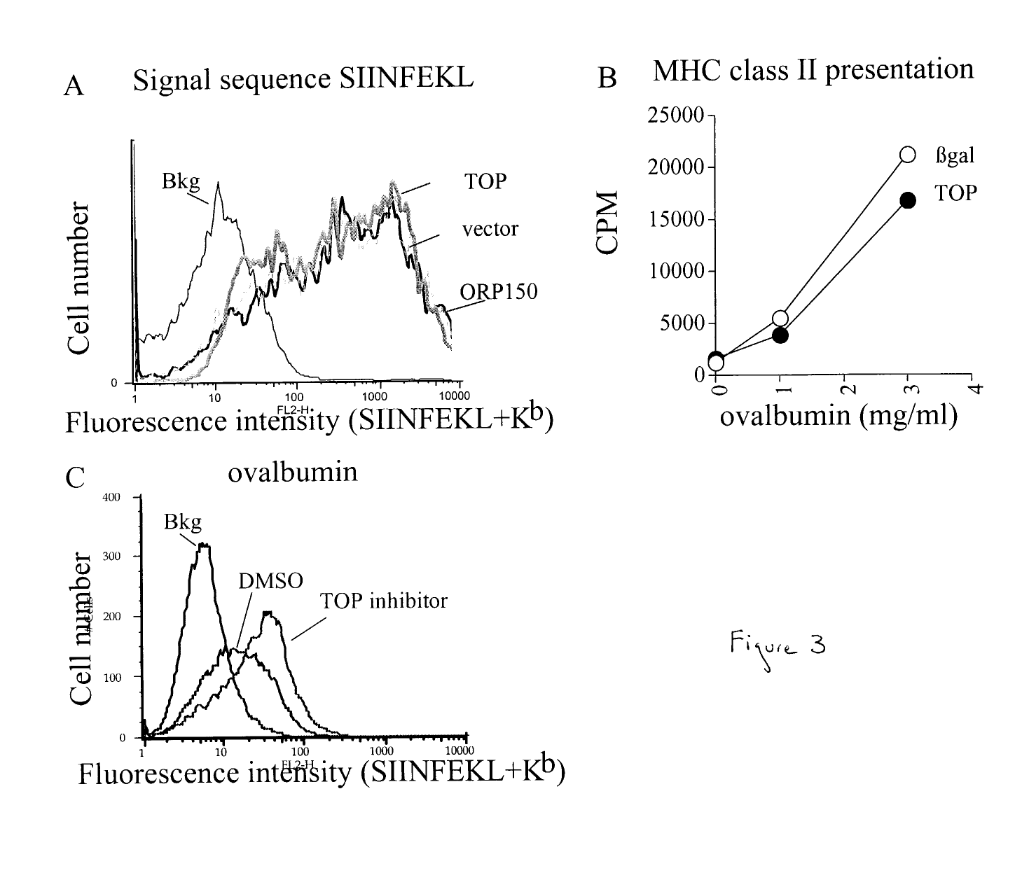

The same experimental approach was used to examine whether the presentation of SIINFEKL from whole ovalbumin, which requires cleavage by proteasomes, was affected by the expression of TOP. The experiments illustrated in FIGS. 2A to 2D show that TOP transfection inhibits MHC class I presentation of a full-length protein.

The data of FIG. 2A were generated using methods similar to those used for FIG. 1A except cells were transfected with pCDNA3 encoding a full-length ovalbumin cDNA instead of the SIINFEKL minigene. These data come from the same experiment as in FIG. 1A. FIG. 2B is similar to FIG. 1B except cells were transfected for two hours with full-length ovalbumin cDNA (5 μg) instead of SIINFEKL. These data come from different groups of the same experiment done on the same day as the data shown in FIG. 1B.

The data of FIG. 2C were generated using methods similar to those used for FIG. 1C except cells were transfected for 4 hours with full-length ovalbumin cDNA (5 μg) instead of KVVRFDKL. These data come from the same experiment as in FIG. 1C.

The data of FIG. 2D were generated using methods similar to those used for FIG. 1D except that the cells were co-infected for six hours with vaccinia recombinants encoding TOP or Bgal and full-length influenza nucleoprotein instead of ASNENMETM.

These experiments demonstrated that TOP overexpression inhibited the presentation of SIINFEKL from ovalbumin in Kb-Cos7 (FIG. 2A) and Kb-E36 (FIG. 2B) cells. In addition, transfection of TOP reduced the presentation of the KVVRFDKL subdominant epitope from ovalbumin (FIG. 2C). In similar experiments, TOP expression also inhibited the presentation of ASNENMETM from full-length influenza nucleoprotein (FIG. 2D). The generation of the KWRFDKL and ASNENMETM peptides like SIINFEKL requires proteasomes. Therefore, cytosolic TOP inhibited the presentation of several antigenic peptides generated by proteasomes from full-length proteins. Such inhibition is useful for suppression of immunogenic responses to cells expressing such TOP activity.

### Example 3

Effect of TOP Levels on Presentation of Antigenic Peptide Directly Delivered to ER

The effect of TOP transfection on the presentation of an antigenic peptide that was delivered directly into the endoplasmic reticulum, by-passing the cytoplasm, was investigated. This was accomplished by expressing in Kb-Cos 7 cells a SIINFEKL minigene with an N-terminal signal sequence, which causes the SIINFEKL peptide to be cotranslationally transported into the endoplasmic reticulum (ER) (Anderson et al., 1991, J. Exp. Med. 174:489-492; Craiu et al., 1997, Proc. Natl. Acad. Sci. USA. 94:10850-10855). This leader sequence then is removed by the signal sequence peptidase. Overexpression of TOP does not inhibit the presentation of the ER-targeted SIINFEKL (FIG. 3A), even though it markedly reduced presentation of SIINFEKL generated in the cytoplasm (FIGS. 1A and 2A). Thus, TOP transfection specifically inhibits the presentation of peptides generated in the cytoplasm but does not affect other steps needed for class I antigen presentation such as MHC class I synthesis, assembly or exocytosis.

### Example 4

Effect of TOP on MHC Class II Antigen

In the MHC class II pathway, peptides are generated by proteolysis in the endocytic compartment (Germain and Margulies, 1993, Annu. Rev. Immunol. 11:403-450), which does not contain TOP. In these experiments L cells expressing the MHC class II molecule, I-Ad, and the p41 isoform of invariant chain were infected with vaccinia recombinants expressing TOP or Bgal and incubated with different concentrations of ovalbumin added to the medium. After four hours the cells were fixed, and the ovalbumin-derived peptides presented on I-Ad were measured with a specific T cell hybridoma.

The data of FIG. 3A were generated in experiments similar to those used for FIG. 1A except that the cells were transfected with a SIINFEKL minigene with an N-terminal signal sequence instead of the unmodified SIINFEKL minigene. These data come from the same experiment as in FIG. 1A.

The data of FIG. 3B were generated by incubating I-Ad+p41 invariant chain expressing L cells (Peterson and Miller, Nature 357, 596-8(1992) with various concentrations of ovalbumin (indicated in the FIG. 3B) for four hours and then fixing the cells with paraformaldehyde. The presence of ovalbumin+I-Ad complexes was detected by measuring the production of IL-2 from the DO 11.10 hybridoma (Shimonkevitz et al. J Exp. Med. 158:303-316 (1983)).

The data of FIG. 3C were generated in experiments in which ovalbumin (40 mg/ml) admixed with either the TOP inhibitor cPP-AAF-pAB (1 mM) or vehicle (DMSO) was introduced into pTracer-transfected Kb-Cos7 cells. After two hours the presence of SIINFEKL+Kb complexes was determined by immunofluorescence as described for FIG. 1A (supra). The background group (Bkg in FIG. 3C) represents cells that were not loaded with ovalbumin.

Overexpression of TOP had little effect on the presentation of the class II-presented peptides generated from ovalbumin (FIG. 3B) even though in L cells it strongly inhibited the presentation on class I molecules of SIINFEKL from full-length ovalbumin or a minigene. Thus, these experiments show that inhibition of TOP enhances MHC class I presentation in normal cells and overexpression of TOP does not reduce the presentation of peptides generated in the endoplasmic reticulum or endocytic compartments. This demonstrates the specificity of TOP modulation for affecting MHC class I antigen presentation.

### Example 5

Effect of TOP Levels on Antigenic Peptide Presentation

To investigate whether the levels of TOP normally present in cells hydrolyzes antigenic peptides and thus limits antigen presentation, we tested whether the highly selective TOP inhibitor, N-(1(RS)-carboxy-3-phenylpropyl)-Ala-Ala-Tyr-p-aminobenzoate (cPP-AAF-PAB) (Orlowski et al., 1988, Biochemistry 27, 597-602), would increase antigen presentation. Since this agent does not enter living cells, it was loaded together with ovalbumin into the cytoplasm of Kb-Cos7 cells by hypertonic lysis of pinosomes (Moore et al., 1988, Cell 54, 777-785). Inhibiting TOP enhanced the generation of SIINFEKL-Kb complexes in cells that were not overexpressing this peptidase (FIG. 3C) and in cells transfected with TOP. This result, which was precisely opposite to that of increasing TOP activity in cells (see FIGS. 1 and 2) strongly suggests that most of the SIINFEKL (or SINFEKL-containing peptides) released by proteasomes are degraded by TOP. Thus, specific modulation of TOP is useful for regulation of MHC class I antigen presentation.

### Example 6

Effect of TOP Transfection on Total Expression of MHC Class I Molecules

Newly synthesized MHC class I molecules are retained in the endoplasmic reticulum until they bind peptides (York and Rock, 1996, Annu. Rev. Immunol. 14:369-396). Consequently, conditions that limited the supply of peptides to class I molecules (e.g. inhibition of the proteasome or the TAP transporter) reduced MHC appearance on the cell surface (Rock et al., 1994, Cell 78, 761-771; Townsend and Trowsdale, 1993, Seminars Cell Biol. 4:53-61). If TOP is destroying large numbers of antigenic peptides, there should be a reduction in class I molecules on the cell surface. To test this, cDNAs for a class I molecule were co-transferred into Cos7 cells together with TOP or a control vector. After 48 hours the expression of the class I molecule on the transfected cells was measured by flow cytometry.

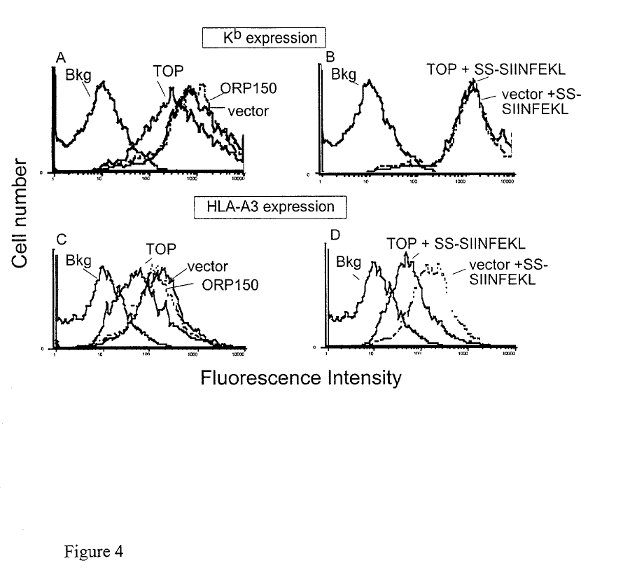

FIGS. 4A to 4D show that TOP inhibits the appearance of MHC class I molecules on the cell surface. The data shown in FIG. 4A were generated in experiments in which Cos7 cells were co-transfected with Kb in pcDNA3 and a pTracer plasmid encoding GFP or TOP and GFP. The Kb transfection was omitted in the background group. After 48 hours the cells were stained with anti-Kb (Y3) and phycoerythrin-anti-Ig antibodies and analyzed by flow cytometry as described in FIG. 1A. The data shown in FIG. 4B were generated in experiments similar to those used to generate the data of FIG. 4A except cells were also transfected with a SIINFEKL minigene.

The experiments in which the data depicted in FIG. 4C were generated were similar to those used to generate the data of FIG. 4A except that the cells were transfected with HLA-A3 instead of Kb and were stained with anti-HLA-A3 instead of anti-Kb.

The experiments in which the data depicted in FIG. 4D were generated were similar to those used to generate the data of FIG. 4C except cells were also transfected with a SIINFEKL minigene. All panels shown in these FIGS. are from the same experiment.

Overexpression of TOP caused a marked reduction in the levels of Kb (FIG. 4A) and HLA-A3 (FIG. 4C) molecules on the cell surface. In contrast, transfection of TOP did not reduce the surface expression of transfected influenza hemaglutinin, a membrane protein whose expression is not dependent on antigenic peptides. To confirm that this decrease in surface class I molecules was due to a reduction in the supply of antigenic peptides, we coexpressed the leader peptide-SIINFEKL construct (which by-passes TOP destruction in the cytoplasm) in these same cells. In this situation, levels of Kb (which binds SINFEKL) on the cell surface are not reduced by TOP (FIG. 4B), whereas the levels of HLA-A3 (which does not bind SIINFEKL) remain low (FIG. 4D).

### Example 7

Bone Marrow Stromal Cells in Ex Vivo Gene Therapy

TOP overexpression can be used to suppress MHC class I antigen presentation in animals having such systems, e.g., mammals, including dogs and humans.

For example, in dogs, bone marrow stromal cells are cultured in vitro from primary iliac crest marrow aspirates of dogs. Cell cultures are expanded to greater than 1×10⁸ cells in vitro using a suitable medium. The cells of the dogs are transduced ex vivo by transfection (e.g., Lipofection or calcium phosphate) with a plasmid expression vector, comprising the TOP gene, and suitable enhancers (e.g., SV40) or promoters (e.g., thymidine kinase). Levels of TOP expression in the transfected cells of each of the dogs can be tested in vitro in tissue culture media, and can be determined by any suitable assay method, for example, radioimmunoassay techniques. The ex vivo modified bone marrow stromal cells are reintroduced into each dog in an autologous manner, for example, by reinfusion into one foreleg vein or by direct intracavitary injection into the iliac crest bone marrow cavity. TOP gene expression, and/or antigen presentation into dog peripheral blood plasma from reintroduced ex vivo modified stromal cells are measured following reintroduction of cells into dog.

Similar methods can be used in humans. Although the cells can be heterologous, autologous cells are generally preferred. In addition, the bone marrow stromal cells can also be transduced with a desired therapeutic gene using methods known in the art.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A recombinant cell comprising an exogenously derived nucleic acid coding for a thimet oligopeptidase (TOP) polypeptide, such that the TOP polypeptide is overexpressed in the cell compared to a wild-type cell from which the recombinant cell was derived.

2. The cell of claim 1, wherein the nucleic acid coding for the TOP polypeptide is a heterologous nucleic acid sequence.

3. The cell of claim 1, wherein the TOP polypeptide is a full-length TOP polypeptide.

4. The cell of claim 1, wherein the sequence is introduced into the cell by transfection with a vector.

5. The cell of claim 1, wherein the cell was transfected by a viral vector.

6. The cell of claim 1, wherein the cell is a stem cell.

7. A method for reducing the antigenicity of a cell, the method comprising introducing into the cell a nucleic acid that expresses a TOP polypeptide.

8. The method of claim 6, wherein the cell is a stem cell.

9. A method for modulating an antigenic response in a mammal, the method comprising administering to the mammal cells transfected with a nucleic acid encoding a TOP polypeptide.

10. The method of claim 9, wherein the cell is a stem cell.

11. The method of claim 9, wherein the cell is removed from the mammal, transfected in vitro, and reintroduced into the mammal.

12. A method of screening a test compound for its ability to serve as an immunomodulatory agent, the method comprising
(a) obtaining a test compound;
(b) contacting the test compound with a thimet oligopeptidase (TOP); and
(c) assessing the ability of the test compound to interact with TOP, wherein a test compound that interacts with TOP is a candidate immunomodultory agent.

13. The method of claim 12, wherein the interaction is direct binding of the test compound to TOP.

14. The method of claim 12, wherein the test compound interacts with TOP by altering TOP expression or activity.

15. The method of claim 14, wherein TOP expression or activity is increased.

16. The method of claim 14, wherein TOP expression or activity is decreased.

17. The cell of claim 1, wherein the cell overexpresses TOP and puromycin-sensitive aminopeptidase (PSA).

18. The cell of claim 1, wherein the cell expresses an amount of TOP effective to reduce the level of one or more MHC class I-presented antigenic peptides present in the cell.

19. The cell of claim 1, wherein the cell expresses an amount of TOP effective to reduce the T-cell response otherwise produced in a mammal to the cell.

20. The cell of claim 1, wherein the cell was transfected with a vector comprising a nucleic acid encoding PSA, such that PSA is overexpressed in the cell.

21. The cell of claim 20, wherein the vector is a viral vector.

22. A method of modulating an antigenic response to a cell in a mammal, the method comprising inducing overexpression of TOP in the cell and introducing the cell into the mammal.

23. The method of claim 22, wherein PSA is overexpressed.

24. The method of claim 22, wherein the cell is transfected with a nucleic acid encoding a TOP protein or polypeptide.

25. The method of claim 24, wherein the transfected mammalian cell is a stem cell.

26. A method for modulating an antigenic response in a mammal, the method comprising administering to cells or tissue of a mammal in vivo a composition comprising a nucleic acid encoding a TOP polypeptide.

27. A method for modulating an antigenic response in a mammal, the method comprising administering to the mammal an effective amount of a TOP inhibitor to the mammal.

28. The method of claim 27, further comprising the step of administering to the mammal cells comprising enhanced levels of antigenic peptides.

29. The method of claim 27, wherein at least two inhibitors of TOP are administered to the mammal.

30. The method of claim 27, further comprising administering a PSA inhibitor to the mammal.

31. A method for identifying an antigen resistant to thimet oligopeptidase (TOP) degradation, the method comprising
(i) selecting a first antigen that is degraded by TOP;
(ii) synthesizing a second antigen homologous to the first antigen except for one or more amino acid substitutions, additions, or deletions relative to the first antigen; and
(iii) assessing the degradation of the second antigen to thimet oligopeptidase degradation compared to degradation of the first antigen, wherein a decrease in the amount of degradation of the second antigen compared to the amount of degradation of the first antigen indicates that the second antigen is resistant to TOP degradation.

32. The method of claim 31, wherein the second antigen binds MHC Class I molecules or T-cell receptors.

33. A tissue engineering construct comprising a cell of claim 1.

34. A method of screening test compounds for their ability to modulate thimet oligopeptidase (TOP) expression or activity, the method comprising
(i) obtaining a test compound;
(ii) contacting the test compound with TOP or a nucleic acid that encodes TOP; and
(iii) assessing the ability of the test compound to interact with TOP or the nucleic acid that encodes TOP, wherein a test compound that interacts with TOP or a TOP-encoding nucleic acid is a candidate immunomodulatory agent.

35. A method of increasing CD8 T cell immunity, the method comprising the step of including in a vaccination a TOP inhibitor in an amount effective to decrease TOP expression or activity.

36. The method of claim 35, wherein the vaccination method uses treated tumor cells, antigen bearing/pulsed dendritic cells, or injection of a viral vector.
